(19) United States
(12) Patent Application Publication
(10) Pub. No.: US 2025/0195551 A1
(43) Pub. Date: Jun. 19, 2025

(54) LIQUID FORMULATIONS OF IVERMECTIN COMPOSITIONS AND USE IN GELATIN DOSAGE FORMS

(71) Applicants: L4 BIO LLC, New York, NY (US); Catalent Pharma Solutions, LLC, Somerset, NJ (US)

(72) Inventors: Samuel D. Waksal, New York, NY (US); Rui Wu, Wilmington, DE (US); Wai Sheung Ma, Somerset, NJ (US); Yafei Lyu, Somerset, NJ (US)

(21) Appl. No.: 18/849,392

(22) PCT Filed: Mar. 22, 2023
(86) PCT No.: PCT/US2023/064796

§ 371 Date: Sep. 20, 2024

(51) Int. Cl. A61K 31/7048; A61K 47/14; A61K 47/22; A61K 47/26

(52) U.S. Cl. CPC A61K 31/7048; A61K 47/14; A61K 47/22; A61K 47/26

(57) ABSTRACT

Disclosed are formulations and dosage forms of avermectins, and particularly of ivermectin. The disclosed compositions may be used in methods for the treatment and prevention of various neurological and other disorders in humans.

### FIELD

The present disclosure provides compositions and dosage forms of avermectins, and particularly of ivermectin. The compositions may be used for the treatment and prevention of various neurological and other disorders.

### BACKGROUND

The avermectins are a family of 16-membered macrocyclic lactone derivatives with potent anthelmintic and insecticidal properties and are used as active agents for the treatment or prevention of infection by parasitic worms and other parasitic infections. Avermectins are a series of macrolides, each of which is substituted thereon at the 13-position with a 4-(α-L-oleandrosyl)-α-L-oleandrose group. Avermectins are produced by cultures of the bacterium Streptomyces avermitilis or by synthetic or semi-synthetic means. The members of the avermectin family bind selectively and with high affinity to glutamate-gated chloride ion channels, which occur in invertebrate nerve and muscle cells. This leads to an increase in the permeability of the cell membrane to chloride ions with hyperpolarization of the nerve or muscle cell, resulting in paralysis and death of the parasite. All avermectin family of compounds show a similar spectrum of activity in different level of potency.

Ivermectin, an avermectin family member, is a highly potent anti-parasitic agent. Ivermectin is a mixture of 5-O-demethyl-22,23-dihydroavermectin A1a (also called 22,23-dihydroavermectin B1a) and 5-O-demethyl-25-de(1-methylpropyl)-22,23-dihydro-25-(1-methylethyl)avermectin A1a (also called 22,23-dihydroavermectin B1b). Ivermectin has been used historically as a broad-spectrum anti-parasitic medicinal product for human and veterinary use.

Ivermectin is commercially available for animal use as Cardomec (for felines), Eqvalane (for equines) and Ivomec (for bovines) by Merial; as Zimecterin (for equines) by Famam Companies, Inc. The medicine is available in tablets and chewables for heartworm prevention, topical solution for ear mite treatment, and injectable solution, oral paste or solution for other parasites in veterinary use. Ivermectin is also available for human use for treating parasitic infestations. For example, Stromectol, containing 3 mg ivermectin/tablet and marketed by Merck & Co., is approved by the U.S. Food and Drug Administration to treat onchoceriasis (river blindness) and strongyloidiasis (non-disseminated intestinal threadworm). Ivermectin may exert its antiparasitic activity via activation of a chloride ion-gated glutamate channel present in the invertebrate nervous system. Binding to the chloride ion-gated glutamate channel may result in hyperpolarization of nerves and muscle fiber. Such hyperpolarization may lead to paralysis and death of the organism (parasite). The chloride ion-gated glutamate channels are specific for invertebrates and are not expressed in the mammalian hosts, allowing for a specific action of ivermectin to be directed at the parasites.

The currently used medications for neurological disorders featuring seizure or movement disorders show significant side effects such as behavioral changes, lethargy, insomnia, clinical depression, psychotic behavior, respiratory depression, coma and death, particularly when taken in overdoses or for long periods of time. Thus, there is an unmet need for development of alternative drugs that can be used to treat or prevent such neurological and other disorders that cause little or no adverse health risks.

### SUMMARY

The disclosure provides a pharmaceutical composition comprising:
(i) about 1% to about 15% of ivermectin comprising a compound of Formula VI (22,23-dihydroavermectin B1a) and a compound of Formula VII (22,23-dihydroavermectin B1b);
(ii) about 20% to about 40% of a first surfactant comprising one or more of:
   (a) mono-, di-, and/or tri-fatty acid esters of glycerol;
   (b) mono- and/or di-fatty acid esters of 1,2-propylene glycol; and
   (c) mono- and/or di-fatty acid esters of polyethylene glycol wherein the fatty acids are selected from C₆ to C₁₀ fatty acids; and
(iii) about 15% to about 70% of a second surfactant selected from one or more of a polysorbate surfactant and/or a fatty acid ester of sorbitan.

In some embodiments, the pharmaceutical composition comprises ivermectin comprising at least about 70% of 22,23-dihydroavermectin B1a and less than about 30% of 22,23-dihydroavermectin B1b. In some embodiments, the pharmaceutical composition comprises ivermectin comprising at least about 90% of 22,23-dihydroavermectin B1a and less than about 10% of 22,23-dihydroavermectin B1b.

In embodiments, the pharmaceutical composition comprises about 3% to about 12% of a compound of any one of Formulas I-VII. In some embodiments, the pharmaceutical composition comprises about 5% to about 10% of a compound of any one of Formulas I-VII. In some embodiments, the pharmaceutical composition comprises about 1%, about 2%, about 3%, about 4%, about 5%, about 6%, about 7%, about 8%, about 9%, about 10%, about 11%, or about 12% of a compound of any one of Formulas I-VII.

In some embodiments, the pharmaceutical composition comprises about 3% to about 12% of one or more of a compound of Formulas I-VII. In some embodiments, the pharmaceutical composition comprises about 5% to about 10% of one or more of a of Formulas I-VII. In some embodiments, the pharmaceutical composition comprises about 1%, about 2%, about 3%, about 4%, about 5%, about 6%, about 7%, about 8%, about 9%, about 10%, about 11%, or about 12% of one or more of a of Formulas I-VII.

In some embodiments, the pharmaceutical composition comprises about 3% to about 12% of ivermectin comprising a compound of Formula VI and a compound of Formula VII. In some embodiments, the pharmaceutical composition comprises about 5% to about 10% ivermectin comprising a compound of Formula VI and a compound of Formula VII. In some embodiments, the pharmaceutical composition comprises about 1%, about 2%, about 3%, about 4%, about 5%, about 6%, about 7%, about 8%, about 9%, about 10%, about 11%, or about 12% ivermectin comprising a compound of Formula VI and a compound of Formula VII.

In embodiments, the pharmaceutical composition includes a first surfactant, wherein the fatty acids are selected from C₈ to C₁₀ fatty acids. In some embodiments, the first surfactant comprises mono- and di-fatty acid esters of glycerol. In some embodiments, the first surfactant is selected from Masester M8120, Capryol 90, Labrasol ALF, and combinations thereof. In some embodiments, the pharmaceutical composition includes about 25% to about 30% of the first surfactant.

In embodiments, the pharmaceutical composition includes a second surfactant selected from polysorbate 80 (Tween 80), sorbitan monolaurate (Span 20), and combinations thereof. In some embodiments, the pharmaceutical composition includes about 15% to about 20% of the second surfactant. In some embodiments, the pharmaceutical composition includes about 30% to about 35% of the second surfactant. In some embodiments, the pharmaceutical composition includes about 60% to about 65% of the second surfactant.

In some embodiments, the pharmaceutical composition comprises about 5% to about 55% D-α-Tocopherol polyethylene glycol 1000 succinate (vitamin E TPGS); or about 30% to about 50% vitamin E TPGS. In an embodiment, the pharmaceutical composition is in a dosage form comprising a gelatin capsule.

In embodiments, the pharmaceutical composition comprises: (i) about 5% to about 10% ivermectin; (ii) about 25% to about 30% mono- and di-C₈ to C₁₀ fatty acid esters of glycerol; (iii) about 15% to about 30% of a polysorbate surfactant; and (iv) about 30% to about 50% vitamin E TPGS.

In an embodiment, the pharmaceutical composition comprises: (i) about 8% ivermectin; (ii) about 27.6% mono- and di-C₈ to C₁₀ fatty acid esters of glycerol; (iii) about 32.2% polysorbate 80; and (iv) about 32.2% vitamin E TPGS.

In an embodiment, the pharmaceutical composition comprises: (i) about 8% ivermectin; (ii) about 27.6% Masester E8120; (iii) about 32.2% polysorbate 80; and (iv) about 32.2% vitamin E TPGS.

In an embodiment, the pharmaceutical composition comprises: (i) about 8% ivermectin; (ii) about 27.6% mono- and di-C₈ to C₁₀ fatty acid esters of glycerol; (iii) about 18.4% polysorbate 80; and (iv) about 46% vitamin E TPGS.

In an embodiment, the pharmaceutical composition comprises: (i) about 8% ivermectin; (ii) about 27.6% Masester E8120; (iii) about 18.4% polysorbate 80; and (iv) about 46% vitamin E TPGS.

In an embodiment, the pharmaceutical composition comprises: (i) about 8% ivermectin; (ii) about 27.6% mono- and di-C₈ to C₁₀ fatty acid esters of glycerol; and (iii) about 64.4% polysorbate 80.

In an embodiment, the pharmaceutical composition comprises: (i) about 8% ivermectin; (ii) about 27.6% Masester E8120; and (iii) about 64.4% polysorbate 80.

### BRIEF DESCRIPTION OF THE FIGURES

FIG. 1. Graph showing fiberoptic dispersion of ivermectin formulation prototypes in FaSSIF (1E-7E).

FIG. 2. The mean concentration-time curve of ivermectin B1a in SD rat plasma after a single fasting intravenous injection with 2 mg/kg ivermectin API.

FIG. 3. The mean concentration-time curve of ivermectin B1a in SD rat plasma after a single fasting oral administration with 2 mg/kg Stromectol.

FIG. 4. The mean concentration-time curve of ivermectin B1a in SD rat plasma after a single fasting oral administration with 2 mg/kg Ivomec.

FIG. 5. The mean concentration-time curve of ivermectin B1a in SD rat plasma after a single fasting oral administration with 2 mg/kg formulation 7E.

FIG. 6. Schematic showing the study to evaluate modulation of pentylenetetrazol (PTZ)-induced seizures by a liquid solution of formulation 7E in rats.

FIG. 7. Area under the curve values from the BOLD fMRI timeseries from selected brain regions. Data are presented as mean+SEM, n=12 per group. From left to right: (1) Vehicle+PTZ, (2) IVM liquid solution, 2 mg/kg+PTZ, (3) IVM liquid solution, 4 mg/kg+PTZ, Alprazolam (3 mg/kg)+PTZ. Statistical significances: (p<0.05, p<0.01, p<0.001, p<0.0001, Ordinary two-way ANOVA). Vehicle vs. Alprazolam in all; Vehicle vs. IVM (4 mg/kg+PTZ) in all except in HipAD and Pons. Alprazolam vs. IVM 2 mg/kg in CinCtx, MotCtx, RSCtx, SSCtx, ThaIVM & mPFC. CC=Corpus Callosum; CinCtx=Cortex Cingulate; MotCtx=Motor Cortex; RSCtx=Retrosplenial Cortex; SSCtx=Somatosensory Cortex; TAssocCtx=Temporal Association Cortex; HipAD=Hippocampus Antero Dorsal; Pons; ThalDL=Dorsolateral Thalamus; ThalVM=Ventromedial Thalamus; VTA=Ventral Tegmental Area; nAcbSh=Nucleus of the Accumbens Shell; Amygdala; CPu=Caudate Putamen; and mPFC=medial prefrontal cortex.

### DETAILED DESCRIPTION

The present disclosure provides pharmaceutical compositions and dosage forms of avermectins, and particularly of ivermectin. The compositions (also referred herein interchangeably as formulations) of the avermectins are liquid or semi-solid at room temperature and comprise an avermectin, and particularly ivermectin, a first surfactant and a second surfactant. The compositions may be used for the treatment and prevention of neurological and other disorders.

### Avermectins and Ivermectin

Any of the avermectin compounds, which includes derivatives and analogs disclosed herein, may be used in the compositions and methods provided by this disclosure. Avermectins include a family of four closely related major components, A1a, A2a, B1a and B2a and four minor components A1b, A2b, B1b, B2b which are lower homologs of the corresponding major components. Eight different avermectins were isolated in four pairs of homologue compounds, with a major and minor component usually in ratios of about 80:20 to about 90:10. Anthelmintics derived from the avermectins include ivermectin, selamectin, doramectin, eprinomectin, moxidectin, avermectin, and abamectin. The family members show anthelmintic and insecticidal/acaricidal activity in different degree of potencies.

Provided herein are compositions comprising about 1% to about 15% of an avermectin compound, wherein the avermectin compound may be a macrocyclic compound according to Formula I:

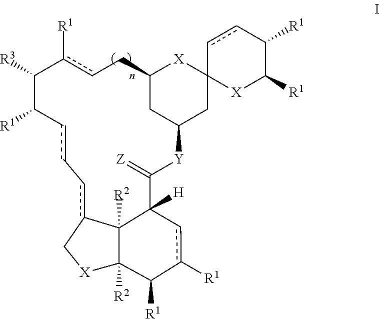

or a pharmaceutically acceptable salt, stereoisomer, polymorph, solvate, or isotopically labeled compound thereof, wherein:
each occurrence of X is independently selected from —CH₂—, —NH—, —O—, —S—, —SO— and —SO₂—;
Y is selected from —CH₂—, —O—, —NH—, and —S—;
Z is selected from O and S;
each occurrence of [symbol] is a single bond or a double bond;
n is an integer 0-6; and
each occurrence of R¹ is independently selected from halogen, —R, —OR, —NO₂, —NCS, —CN, —CF₃, —OCF₃, —NHR, —N(R)₂, —OC(O)R, —C(O)OR, —SR, —C(O)R, —C(O)C(O)R, —C(O)CH₂C(O)R, —C(S)R, —C(S)OR, —C(O)C(O)OR, —C(O)C(O)N(R)₂, —C(O)N(R)₂, —OC(O)N(R)₂, —C(S)N(R)₂, —N(R)C(O)OR, —N(R)C(O)R, —N(R)C(S)R, —N(R)C(O)N(R)₂, —N(R)C(S)N(R)₂, —N(COR)COR, —N(OR)R, —C(═NH)N(R)₂, —C(O)N(OR)R, —C(═NOR)R, —OP(O)(OR)₂, —P(O)(R)₂, —P(O)(OR)₂, —P(O)(H)(OR), —CH₂—OR, and —CH₂—O—CH₂—R;
each occurrence of R² is independently selected from independently selected from H, OH, O—C₁₋₄alkyl, —OC(O)C₁₋₄alkyl, —OC(O)NH₂, and —OC(O)NHC₁₋₄alkyl;
each occurrence of R³ is mono, di, or triglycoside, or OC(O)—(C₃-C₅)alkenyl;
each R is independently selected from H, —(C₁-C₁₂)alkyl, —(C₃-C₁₀)-cycloalkyl, (C₃-C₁₀)-cycloalkenyl, —[(C₃-C₁₀)cycloalkyl]-(C₁-C₁₂)alkyl, —[(C₃-C₁₀)cycloalkenyl]-(C₁-C₁₂)alkyl, —[(C₃-C₁₀)cycloalkenyl]-(C₁-C₁₂)alkyl, —[(C₃-C₁₀)cycloalkyl]-O—(C₁-C₁₂)alkyl, —[(C₃-C₁₀)cycloalkenyl]-O—(C₁-C₁₂)alkyl, —(C₆-C₁₀)aryl, (C₆-C₁₀)aryl-(C₁-C₁₂)alkyl, —(C₆-C₁₀)aryl-O—(C₁-C₁₂)alkyl, (C₆-C₁₀)aryl-N(R″)—(C₁-C₁₂)alkyl, 3- to 10-membered heterocyclyl, (3- to 10-membered heterocyclyl)-(C₁-C₁₂)alkyl, (3- to 10-membered heterocyclyl)-O—(C₁-C₁₂)alkyl, (3- to 10-membered heterocyclyl)-N(R″)—(C₁-C₁₂)alkyl, 5- to 10-membered heteroaryl, (5- to 10-membered heteroaryl)-(C₁-C₁₂)-alkyl, (5- to 10-membered heteroaryl)-O—(C₁-C₁₂)-alkyl and (5- to 10-membered heteroaryl)-N(R″)—(C₁-C₁₂)-alkyl;
   each heterocyclyl has 1-4 heteroatoms independently selected from N, NH, O, S, SO, and SO₂, and
   heteroaryl has 1-4 heteroatoms independently selected from N, NH, 0, and S;
   each occurrence of R is independently unsubstituted or is substituted with 1 to 5 R′;
   each occurrence of R′ is halo, OH, oxo, —CH₂OR″, —CH2N(R″)₂, C(O)N(R″)₂, —C(O)OR″, —NO₂, NCS, CN, CF₃, OCF₃ and —N(R″)₂; and
   each occurrence of R″ is independently H, C₁₋₆alkyl, C₂₋₆alkenyl, C₃₋₆cycloalkyl, C₃₋₆cycloalkenyl, 3- to 6-membered heterocyclyl, 5- to 10-membered heteroaryl, and (C₆-C₁₀)-aryl.

In some embodiments, the compound of Formula I is a compound of Formula II:

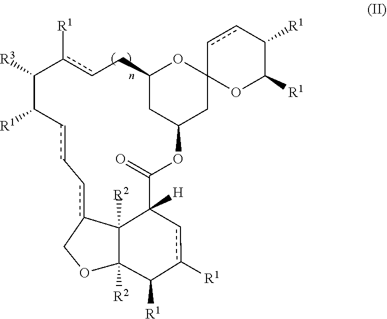

or a pharmaceutically acceptable salt, stereoisomer, polymorph, solvate or isotopically labeled compound thereof, wherein n, R¹, R², and R³ are each as defined in Formula I.

In some embodiments, the compound of Formula II is a compound of Formula III:

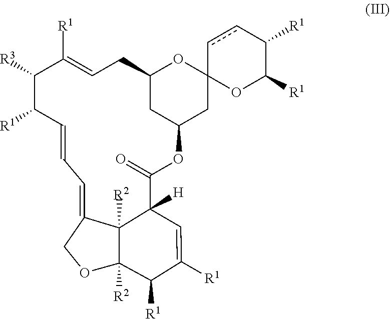

or a pharmaceutically acceptable salt, stereoisomer, polymorph, solvate or isotopically labeled compound thereof, wherein R¹, R², and R³ are each as defined in Formula I.

In some embodiments, the compound of Formula III is a compound of Formula IV:

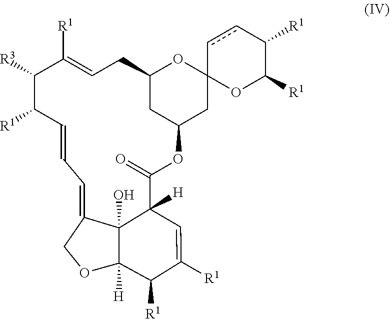

or a pharmaceutically acceptable salt, stereoisomer, polymorph, solvate or isotopically labeled compound thereof, wherein:
each occurrence of R¹ is independently selected from halogen, —R, —OR, —NO₂, —NCS, —CN, —CF₃, —OCF₃, —NHR, —N(R)₂, —OC(O)R, —C(O)OR, —C(O)N(R)₂, —OC(O)N(R)₂, —N(R)C(O)OR, —N(R)C(O)R, —N(R)C(O)N(R)₂, —CH₂—OR, and —CH₂—O—CH₂—R;
each occurrence of R³ is mono, di, or triglycoside, or OC(O)—(C₃-C₅) alkenyl;
R, R′ and R″ are each as defined in Formula I.

In some embodiments, the compound of Formula IV is a compound of Formula V:

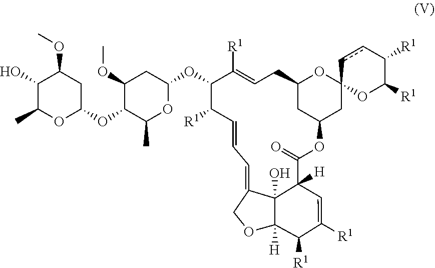

or a pharmaceutically acceptable salt, stereoisomer, polymorph, solvate or isotopically labeled compound thereof, wherein
each occurrence of R¹ is independently selected from halogen, —R, —OR, —NO₂, —NCS, —CN, —CF₃, —OCF₃, —NHR, —N(R)₂, —OC(O)R, —C(O)OR, —C(O)N(R)₂, —OC(O)N(R)₂, —N(R)C(O)OR, —N(R)C(O)R, —N(R)C(O)N(R)₂, —CH₂—OR, and —CH₂—O—CH₂—R:
R, R′ and R″ are each as defined in Formula I.

In embodiments, the macrocyclic compound is an avermectin compound or derivative, a pharmaceutically acceptable salt, ester, stereoisomer, polymorph, solvate, N-oxide, or isotopically labeled compound thereof. In an embodiment, the macrocyclic compound is avermectin. In some embodiments, the macrocyclic compound is selected from the group consisting of ivermectin, selamectin, doramectin, eprinomectin, moxidectin, avermectin, and abamectin. In some embodiments, the macrocyclic compound is ivermectin or a derivative, a pharmaceutically acceptable salt, ester, stereoisomer, polymorph, solvate, N-oxide, or isotopically labeled compound thereof.

Provided herein is a composition comprising about 1% to about 15% of a compound of any one of Formulas I-VII. In some embodiments, the composition comprises about 1% to about 15% of one or more of a compound of any one of Formulas I-VII. In some embodiments, the composition comprises about 1% to about 15% ivermectin comprising 22,23-dihydroavermectin B1a (Formula VI) and 22,23-dihydroavermectin B1b (Formula VII). In some embodiments, the composition comprises ivermectin comprising from at least about 70% to at least about 90% of a compound of Formula VI and from less than about 30% to less than about 10% of a compound of Formula VII:

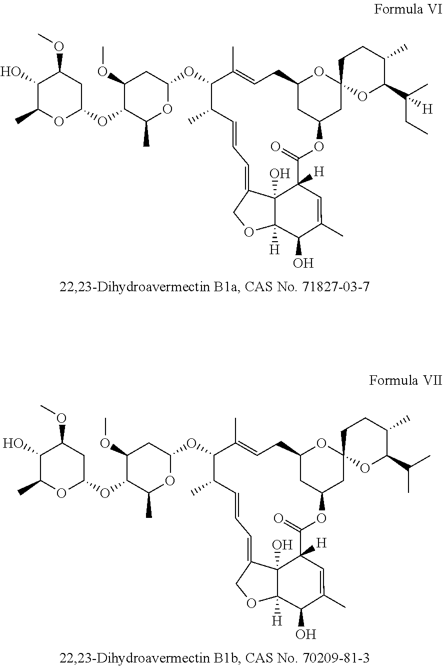 [SMILES: CC[C@H](C)[C@H]1O[C@]2(CC[C@@H]1C)C[C@@H]1C[C@@H](C/C=C(\C)[C@@H](O[C@H]3C[C@H](OC)[C@@H](O[C@H]4C[C@H](OC)[C@@H](O)[C@H](C)O4)[C@H](C)O3)[C@@H](C)/C=C/C=C3\CO[C@@H]4[C@H](O)C(C)=C[C@@H](C(=O)O1)[C@]34O)O2 ; CO[C@H]1C[C@H](O[C@H]2[C@H](C)O[C@@H](O[C@@H]3/C(C)=C/C[C@@H]4C[C@@H](C[C@]5(CC[C@H](C)[C@@H](C(C)C)O5)O4)OC(=O)[C@@H]4C=C(C)[C@@H](O)[C@H]5OC/C(=C\C=C\[C@@H]3C)[C@]54O)C[C@@H]2OC)O[C@@H](C)[C@@H]1O]

In some embodiments, the composition comprises ivermectin comprising from at least about 70% to at least about 90% of a compound of Formula VI and from less than about 30% to less than about 10% of a compound of Formula VII. In some embodiments, ivermectin comprises at least about 70% of 22,23-dihydroavermectin B1a and less than about 30% of 22,23-dihydroavermectin B1b. In some embodiments, ivermectin comprises at least about 90% of 22,23-dihydroavermectin B1a and less than about 10% of 22,23-dihydroavermectin B1b.

Derivatives of ivermectin, including abamectin and doramectin, and prodrugs of ivermectin, may have properties and uses similar to those of ivermectin. Abamectin and doramectin both have a double bond at positions C22-C23 in the structural formula of ivermectin. Additionally, in doramectin, position C25 is substituted at the side chain of a cyclohexyl ring.

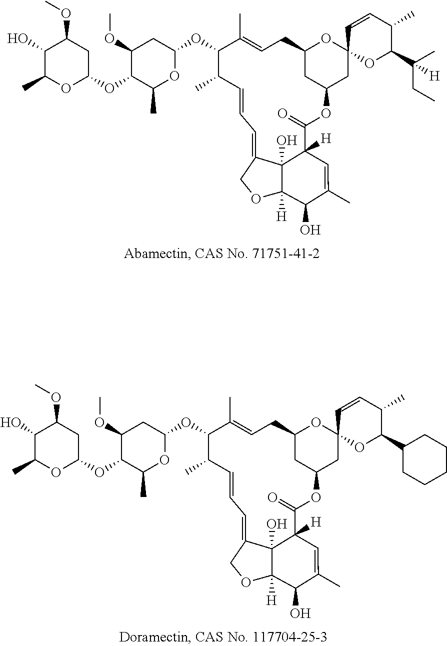 [SMILES: CC[C@H](C)[C@H]1O[C@]2(C=C[C@@H]1C)C[C@@H]1C[C@@H](C/C=C(\C)[C@@H](O[C@H]3C[C@H](OC)[C@@H](O[C@H]4C[C@H](OC)[C@@H](O)[C@H](C)O4)[C@H](C)O3)[C@@H](C)/C=C/C=C3\CO[C@@H]4[C@H](O)C(C)=C[C@@H](C(=O)O1)[C@]34O)O2 ; CO[C@H]1C[C@H](O[C@H]2[C@H](C)O[C@@H](O[C@@H]3/C(C)=C/C[C@@H]4C[C@@H](C[C@]5(C=C[C@H](C)[C@@H](C6CCCCC6)O5)O4)OC(=O)[C@@H]4C=C(C)[C@@H](O)[C@H]5OC/C(=C\C=C\[C@@H]3C)[C@]54O)C[C@@H]2OC)O[C@@H](C)[C@@H]1O]

As used herein, “derivative” to a compound that retains the biological activity of the parent avermectin from which it is derived, or is a prodrug for the parent avermectin. Derivatives may include esters, amides, ethers or the like that are derived from the avermectin.

As used herein, the term “alkyl” refers to a hydrocarbon chain that is a straight chain or a branched chain, containing the indicated number of carbon atoms. For example, C₁₋₆ indicates that the group has from 1 to 6 (inclusive) carbon atoms in it. In the absence of any numerical designation, “alkyl” is a chain (straight or branched) having 1 to 20 (inclusive) carbon atoms in it. The term “unsaturated alkyl” refers to a hydrocarbon having no unsaturated bonds (e.g., a carbon-carbon double bond or a carbon-carbon triple bond). The term “unsaturated alkyl” refers to a hydrocarbon having one or more unsaturated bonds (e.g., a carbon-carbon double bond or a carbon-carbon triple bond).

As used herein, the term “alkenyl” refers to a hydrocarbon chain that is a straight chain or branched chain having one or more carbon-carbon double bonds. The alkenyl moiety contains the indicated number of carbon atoms. For example, C₂₋₆ indicates that the group has from 2 to 6 (inclusive) carbon atoms in it. In the absence of any numerical designation, “alkenyl” is a chain (straight or branched) having 2 to 20 (inclusive) carbon atoms in it.

The term “cycloalkyl” as employed herein includes saturated and partially unsaturated cyclic hydrocarbon groups. Unless indicated otherwise, a cycloalkyl has 3 to 12 carbons, or 3 to 8 carbons, or 3 to 6 carbons, wherein the cycloalkyl group additionally is optionally substituted. Some cycloalkyl groups include, without limitation, cyclopropyl, cyclobutyl, cyclopentyl, cyclopentenyl, cyclohexyl, cyclohexenyl, cycloheptyl, and cyclooctyl.

As used herein, the term “aryl” refers to a 6 to 10 carbon monocyclic or bicyclic aromatic ring system wherein 0, 1, 2, 3, or 4 atoms of each ring are substituted by a substituent. Examples of aryl groups include phenyl, naphthyl and the like.

As used herein, the term “heterocyclyl group” or “heterocyclyl” refers to aromatic and non-aromatic ring compounds containing 3 or more ring members, of which, one or more is a heteroatom such as, but not limited to, N, O, and S. A heterocyclyl can be a cycloheteroalkyl, or a heteroaryl, or if polycyclic, any combination thereof. Heterocyclyl groups may comprise from 3 to about 20 ring members, whereas other such groups may comprise from 3 to about 15 ring members. A heterocyclyl group designated as a C₂-heterocyclyl can be a 5-membered ring with two carbon atoms and three heteroatoms, a 6-membered ring with two carbon atoms and four heteroatoms, and so forth. Likewise, a C₄-heterocyclyl can be a 5-membered ring with one heteroatom, a 6-membered ring with two heteroatoms, and so forth. The number of carbon atoms and the number of heteroatoms, when summed up, equal the total number of ring atoms. A heterocyclyl ring can also include one or more double bonds. A heteroaryl ring is an embodiment of a heterocyclyl group. In some embodiment, the “heterocyclyl” refers to a nonaromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of O, N, or S if monocyclic, bicyclic, or tricyclic, respectively), wherein 0, 1, 2 or 3 atoms of each ring are substituted by a substituent. Examples of heterocyclyl groups include piperazinyl, pyrrolidinyl, dioxanyl, morpholinyl, tetrahydrofuranyl, and the like.

As used herein, the term “heteroaryl” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of O, N, or S if monocyclic, bicyclic, or tricyclic, respectively), wherein 0, 1, 2, 3, or 4 atoms of each ring are substituted by a substituent. Examples of heteroaryl groups include pyridyl, furyl or furanyl, imidazolyl, benzimidazolyl, pyrimidinyl, thiophenyl or thienyl, quinolinyl, indolyl, thiazolyl, and the like.

As used herein, the term “glycoside” refers to any material with a chemical structure comprising a glycosidic bond between a carbohydrate (sugar) molecule and another carbohydrate or a non-carbohydrate (non-sugar) moiety. A glycosidic bond or glycosidic linkage is a type of covalent bond that joins a carbohydrate (sugar) molecule, for example, via its hemiacetal or hemiketal group, to another molecule. The other molecule may or may not be a carbohydrate. The sugar moiety is generally known as the glycone part of a glycoside. The glycone can consist of a single sugar group (monosaccharide), two sugar groups (disaccharide) or several sugar groups (oligosaccharide).

As used herein, the term “substituent” refers to a group replacing a second atom or group such as a hydrogen atom on any molecule, compound or moiety. Suitable substituents include, without limitation, halo, hydroxy, mercapto, oxo, nitro, haloalkyl, alkyl, alkaryl, aryl, aralkyl, alkoxy, thioalkoxy, aryloxy, amino, alkoxycarbonyl, amido, carboxy, alkanesulfonyl, alkylcarbonyl, and cyano groups.

In embodiments, the avermectin used in the formulations and methods provided herein will be in the neutral form. However, when the avermectin comprises an ionizable group, the avermectin may be present as a pharmaceutically acceptable salt. As used herein, the term “pharmaceutically acceptable salt” refers to those salts which are suitable for pharmaceutical use, such as, for example, for use in humans and animals. Pharmaceutically acceptable salts of amines, carboxylic acids, and other types of compounds, are well known in the art. For example, S. M. Berge, et al., describe pharmaceutically acceptable salts in detail in J Pharmaceutical Sciences, 66: 1-19 (1977), incorporated herein by reference. The salts can be prepared in situ during the final isolation and purification of the avermectin, or separately by reacting a free base or free acid function with a suitable reagent. For example, a free base function can be reacted with a suitable acid. Suitable pharmaceutically acceptable salts can, include metal salts such as alkali metal salts, e.g. sodium, potassium, and lithium salts; and alkaline earth metal salts, e.g. calcium or magnesium salts. Examples of pharmaceutically acceptable, nontoxic acid addition salts are salts of an amino group formed with inorganic acids such as hydrochloric acid, hydrobromic acid, hydroiodic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzoate, bisulfate, borate, butyrate, camphorate, camphorsulfonate, citrate, cyclopentanepropionate, digluconate, dodecylsulfate, formate, fumarate, glucoheptonate, glycerophosphate, gluconate, hernisulfate, heptanoate, hexanoate, hydroiodide, 2-hydroxy-ethanesulfonate, lactobionate, lactate, laurate, lauryl sulfate, malate, maleate, malonate, methanesulfonate, nicotinate, nitrate, oleate, oxalate, palmitate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like.

### Avermectin Compositions

The compositions for use in the methods and dosages provided herein are liquid or semi-solid at room temperature and comprise an avermectin, and particularly ivermectin, a first surfactant and a second surfactant.

In embodiments, the composition comprises:
(i) about 1% to about 15% of a compound of any one of Formulas I-VII;
(ii) about 20% to about 40% of a first surfactant comprising one or more of:
   (a) mono-, di-, and/or tri-fatty acid esters of glycerol;
   (b) mono- and/or di-fatty acid esters of 1,2-propylene glycol; and
   (c) mono- and/or di-fatty acid esters of polyethylene glycol wherein the fatty acids are selected from C₆ to C₁₀ fatty acids; and
(iii) about 15% to about 70% of a second surfactant selected from one or more of a polysorbate surfactant and/or a fatty acid ester of sorbitan.

In some embodiments, the composition comprises about 3% to about 12% of a compound of any one of Formulas I-VII. In some embodiments, the composition comprises about 5% to about 10% of a compound of any one of Formulas I-VII. In some embodiments, the composition comprises about 1%, about 2%, about 3%, about 4%, about 5%, about 6%, about 7%, about 8%, about 9%, about 10%, about 11%, or about 12% of a compound of any one of Formulas I-VII.

In embodiments, the composition comprises:
(i) about 1% to about 15% of one or more of a compound of any one of Formulas I-VII;
(ii) about 20% to about 40% of a first surfactant comprising one or more of:
   (a) mono-, di-, and/or tri-fatty acid esters of glycerol;
   (b) mono- and/or di-fatty acid esters of 1,2-propylene glycol; and
   (c) mono- and/or di-fatty acid esters of polyethylene glycol;
   wherein the fatty acids are selected from C₆ to C₁₀ fatty acids; and
(iii) about 15% to about 70% of a second surfactant selected from one or more of a polysorbate surfactant and/or a fatty acid ester of sorbitan.

In some embodiments, the composition comprises about 3% to about 12% of one or more of a compound of Formulas I-VII. In some embodiments, the composition comprises about 5% to about 10% of one or more of a of Formulas I-VII. In some embodiments, the composition comprises about 1%, about 2%, about 3%, about 4%, about 5%, about 6%, about 7%, about 8%, about 9%, about 10%, about 11%, or about 12% of one or more of a of Formulas I-VII.

In embodiments, the composition comprises:
(i) about 1% to about 15% of ivermectin comprising a compound of Formula VI and a compound of Formula VII;
(ii) about 20% to about 40% of a first surfactant comprising one or more of:
   (a) mono-, di-, and/or tri-fatty acid esters of glycerol;
   (b) mono- and/or di-fatty acid esters of 1,2-propylene glycol; and
   (c) mono- and/or di-fatty acid esters of polyethylene glycol;
   wherein the fatty acids are selected from C₆ to C₁₀ fatty acids; and
(iii) about 15% to about 70% of a second surfactant selected from one or more of a polysorbate surfactant and/or a fatty acid ester of sorbitan.

In some embodiments, the composition comprises about 3% to about 12% of ivermectin comprising a compound of Formula VI and a compound of Formula VII. In some embodiments, the composition comprises about 5% to about 10% ivermectin comprising a compound of Formula VI and a compound of Formula VII. In some embodiments, the composition comprises about 1%, about 2%, about 3%, about 4%, about 5%, about 6%, about 7%, about 8%, about 9%, about 10%, about 11%, or about 12% ivermectin comprising a compound of Formula VI and a compound of Formula VII.

The composition provided herein comprises a first surfactant, which may be a mixture of surfactants, and comprise C₆ to C₁₀ fatty acid esters of glycerol, of 1,2-propylene glycol, and/or of polyethylene glycol. The C₆ to C₁₀ fatty acid(s) may be saturated or unsaturated, but is preferably saturated. More preferably, the fatty acid has from 8 to 10 carbons, and particularly may be caprate, caprylate and mixtures thereof. The avermectin composition comprises about 20% to about 40% of the first surfactant, or about 25% to about 30% of the first surfactant.

The first surfactant may comprise mono-, di-, and/or tri-fatty acid esters of glycerol. The fatty acid ester of glycerol may be a mixture of mono- and di-esters of glycerol. The fatty acid component is one or more C₆ to C₁₀ fatty acid(s) and may be saturated or unsaturated, but is preferable saturated. More preferably, the fatty acid has from 8 to 10 carbons, and particularly may be caprate, caprylate and mixtures thereof. The fatty acid esters of glycerol are commercially available and include Capmul 808G EP/NF (glyceryl monocaprylate), Capmul MCM C8 EP/NF (glyceryl monocaprylate), Capmul MCM (glyceryl caprylate/caprate), Masester E8120 (glycerol mono- and di-caprylate/caprate).

Additionally, or alternatively, the first surfactant may comprise mono- and/or di-fatty acid esters of 1,2-propylene glycol. The fatty acid esters of propylene glycol may be a mixture of mono- and di-esters of propylene glycol, and preferably the fatty acid ester of propylene glycol may be a mono-ester of propylene glycol. The fatty acid component is one or more C₆ to C₁₀ fatty acid(s) and may be saturated or unsaturated but is preferable saturated. More preferably, the fatty acid has from 8 to 10 carbons, and particularly may be caprate, caprylate and mixtures thereof. The fatty acid esters of propylene glycol are commercially available and include Capmul PG-8 (propylene glycol monocaprylate), Capryol 90 (propylene glycol monocaprylate), Capryol PGMC (propylene glycol mono- and di-caprylate).

The first surfactant may additionally or alternatively comprise mono- and/or di-fatty acid esters of polyethylene glycol. The fatty acid ester of polyethylene glycol may be a mixture of mono- and di-esters of polyethylene glycol. The fatty acid component is one or more C₆ to C₁₀ fatty acid(s) and may be saturated or unsaturated but is preferable saturated. More preferably, the fatty acid has from 8 to 10 carbons, and particularly may be caprate, caprylate and mixtures thereof. The polyethylene glycol component may have an average molecular weight of from about 200 to about 800, or from about 300 to about 500. The fatty acid esters of propylene glycol are commercially available and include Labrasol ALF (small fraction of mono-, di- and triglycerides and mainly PEG-8 (MW 400) mono- and diesters of caprylic (C₈) and capric (C₁₀) acids).

The compositions provided herein also comprise a second surfactant, which may be a mixture of surfactants, and is selected from polysorbate surfactants, fatty acid esters of sorbitan, and mixtures thereof. The avermectin composition comprises about 15% to about 70% of a second surfactant, about 15% to about 40% of a second surfactant, or about 15% to about 20% of the second surfactant.

The second surfactant may comprise polysorbate surfactants. The polysorbate surfactants are polyethoxylated sorbitan esterified with fatty acids having the following general structure:

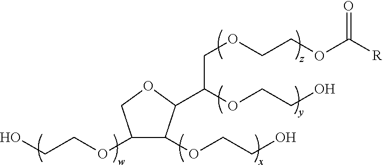

in which R is the carbon chain of a medium to long chain fatty acid, and the sum of w, x, y, and z is the number of oxyethylene —(CH₂CH₂O)— groups found in the molecule. The fatty acid may be saturated or unsaturated. In embodiments, the fatty acid has from 12 to 18 carbons. The number of oxyethylene groups (i.e., w+x+y+z) is in embodiments about 20. Polysorbate surfactants are commercially available and include polysorbate 80 (polyoxyethylene 20 sorbitan monooleate) such as Tween 80, Montanox 80, Alkest TW 80; polysorbate 60 (polyoxyethylene 20 sorbitan monostearate) such as Tween 60; polysorbate 40 (polyoxyethylene 20 sorbitan monopalmitate) such as Tween 40; and polysorbate 20 (polyoxyethylene 20 sorbitan monolaurate) such as Tween 20 and Alkest TW 20.

The second surfactant may additionally or alternatively comprise fatty acid esters of sorbitan (also known as Spans). The fatty acid component may be saturated or unsaturated. In embodiments, the fatty acid has from 12 to 18 carbons. The sorbitan ester may be mono-, di- or tri-esters of sorbitan, and particularly are mono-esters of sorbitan, such as sorbitan monolaurate. Sorbitan esters are commercially available and include Span 20 (sorbitan monolaurate), Span 40 (sorbitan monopalmitate), Span 60 (sorbitan monostearate), Span 65 (sorbitan tristearate) and Span 80 (sorbitan monooleate).

Optionally, the avermectin composition may additionally comprise vitamin E TPGS, also known as D-α-Tocopherol polyethylene glycol 1000 succinate. The composition may comprise from about 5% to about 55% vitamin E TPGS, or from about 30% to about 50% vitamin E TPGS.

In embodiments, the pharmaceutical composition comprises: (i) about 5% to about 10% ivermectin; (ii) about 25% to about 30% mono- and di-C₈ to C₁₀ fatty acid esters of glycerol; (iii) about 15% to about 30% of a polysorbate surfactant; and (iv) about 30% to about 50% vitamin E TPGS.

In an embodiment, the pharmaceutical composition comprises: (i) about 8% ivermectin; (ii) about 27.6% mono- and di-C₈ to C₁₀ fatty acid esters of glycerol; (iii) about 32.2% polysorbate 80; and (iv) about 32.2% vitamin E TPGS.

In an embodiment, the pharmaceutical composition comprises: (i) about 8% ivermectin; (ii) about 27.6% Masester E8120; (iii) about 32.2% polysorbate 80; and (iv) about 32.2% vitamin E TPGs.

In an embodiment, the pharmaceutical composition comprises: (i) about 8% ivermectin; (ii) about 27.6% mono- and di-C₈ to C₁₀ fatty acid esters of glycerol; (iii) about 18.4% polysorbate 80; and (iv) about 46% vitamin E TPGS.

In an embodiment, the pharmaceutical composition comprises: (i) about 8% ivermectin; (ii) about 27.6% Masester E8120; (iii) about 18.4% polysorbate 80; and (iv) about 46% vitamin E TPGS.

In an embodiment, the pharmaceutical composition comprises: (i) about 8% ivermectin; (ii) about 27.6% mono- and di-C₈ to C₁₀ fatty acid esters of glycerol; and (iii) about 64.4% polysorbate 80.

In an embodiment, the pharmaceutical composition comprises: (i) about 8% ivermectin; (ii) about 27.6% Masester E8120; and (iii) about 64.4% polysorbate 80.

The pharmaceutical composition may additionally comprise additional excipients such as additional surfactants, solvents, solubilizing agents, preservatives, anti-oxidants, bulking agents, dissolution enhancers, wetting agents, emulsifiers, suspending agents, antibacterial agents, pH buffering agents, sweeteners, flavoring agents, and combinations thereof.

### Dosage Form

The disclosure provides a composition comprising the avermectin is a semi-solid or liquid-based when formulated as, including, but not limited to, an emulsion, suspension, solution, elixirs, or syrup in which the avermectin is dissolved and/or suspended.

The dosage form comprising the avermectin can take the form of solutions, suspensions, emulsion, aerosols, capsules, soft elastic or hard gelatin capsules, dermal patch, suspensions, and the like preferably in unit dosage forms suitable for simple administration of precise dosages. The composition may take the forms of liquid- or semi-solid filled capsules, sublingual spray, or nasal spray.

Capsule dosage forms may include soft capsules and hard capsules. Capsules may be used as an oral dosage form for the administration of many different types of active pharmaceuticals. The capsules may be filled with an active ingredient in the form of a liquid, or a powder suspended in liquid. Hard capsules can be made of unplasticized or low-plasticized gelatin and water to form a stiff capsule that can be filled with either powder or liquid. Soft capsules can be made of highly plasticized soft elastic gelatin and can contain a liquid or semisolid ingredient. These capsules are often referred to as “softgel” or “gelcap” capsules.

As used herein, the term “capsule” refers to any suitable capsular container or case adapted for oral ingestion, e.g., those adapted for use in conjunction with liquid fill compositions. The term “capsule” may include capsules having a shell composed of soft and/or hard materials, such as gelatin, starches, celluloses, cellulose derivatives (e.g., hydroxypropyl methyl cellulose), hydrocolloids, gums, carrageenans, or any other natural or synthetic material which can be used to encapsulate the liquid composition and be ingested by an animal. Optionally, the shell material can be gelatin and/or hydroxypropyl methyl cellulose. In an embodiment, the shell material is gelatin. The term “capsule” also includes a variety of capsule shapes and sizes. The instant disclosure does not limit the dosage form to a specific type or shape. Any commercially available capsule shells or shell materials can be used.

In an embodiment, the dosage form of the instant disclosure is a soft capsule. In an embodiment, the dosage form of the instant disclosure is a coated liquid-filled soft capsule. The coated capsule can include a liquid fill encapsulated with a soft capsule shell. The exterior surface of the soft capsule shell can be coated with one or more layers of coating.

Suitable materials for encapsulating the liquid fill may include heat sealable polymers and gelatin. Examples of heat sealable polymers may include, but are not limited to, modified starches, cellulosic polymers and carrageenans. In an embodiment, the material is gelatin. The gelatin can be natural gelatin, chemically modified gelatin, enzymatically modified gelatin, or combinations thereof.

The material that forms the capsule shell can further includes water. Water can be present in the original material mass before the capsules are made, in an amount sufficient to allow the processing of the material on the encapsulation machine. After the capsules are formed the majority of the moisture can be removed during the drying process.

The water can have a plasticizing effect on the material. In addition, a non-volatile plasticizer or blend of plasticizers can be added to the material which forms the capsule shell. The non-volatile plasticizer can be any plasticizer compatible with the material of the capsule shell. For example, the non-volatile plasticizer can be glycerin, maltitol, sorbitan, sorbitol or similar low molecular weight polyhydric alcohols, and mixtures thereof. In embodiments, the ratio of plasticizer to material determines how hard or soft the shell can be.

The ratio of plasticizer to material in the shell may be sufficient to provide capsules that are not too hard, such that the capsules are brittle and crack if stressed during shipping and handling, and are not too soft, such that the capsules become deformed during shipping and handling. The non-volatile plasticizer can be present in the capsule shell from about 8% to 65% by total weight of the capsule shell, from about 10% to 35% by total weight of the capsule shell.

The material which forms the capsule shell can further contain extenders. The extender can be any extender which is compatible with the material. Examples of extenders may include natural or modified natural biopolymers and synthetic polymers. Natural biopolymers may include, for instance, cellulose, starch, starch derivatives, bacterial polysaccharides such as xanthan gum and gellan gum and vegetable gums such as guar gum, locust bean gum, gum tragacanth and gum Arabic and animal derived polymers such as chondroitin sulfate, hyaluronic acid, heparin, collagen and chitosan. An example of a modified natural biopolymer may be modified cellulose. Examples of synthetic polymers may include carbon chain polymers of the vinyl and acrylic types as well as heterochains of the polyoxide and polyamine types.

A coating can be applied on the exterior surface of the soft capsule shell. The coating can contain one or more layers. Any coating suitable for a soft capsule can be applied to the capsule. The coating can provide, for example, waterproofing and sealing, smoothing, polishing, enteric protection and/or delayed release properties to the liquid-filled capsule. The delayed release can be affected by, for example, temperature or pH. In an embodiment, the coating is an enteric coating.

The coating can be made by any standard coating ingredient known to those skilled in the art. Coating ingredients may include, but are not limited to, fats, fatty acids, waxes, shellac, ammoniated shellac, cellulose acetate phyhalates, celluosics, vinyls, glycols, acrylics and carbohydrate polymers, polymers and co-polymers containing methacrylic acid and methacrylic acid alkyl esters, hydroxypropylmethyl cellulose (HPMC) and combinations thereof.

The coated capsule can further comprise a finishing coating. In an embodiment, the finishing layer is applied to the coated-capsule. Examples of substance suitable for use in a finishing coating may include, but are not limited to cellulosics, vinyls, glycols, acrylics and carbohydrate polymers and/or combinations thereof.

The liquid fill or semi-solid fill can be encapsulated with a soft capsule shell by any method known in the art. For example, a soft capsule can be made using a standard rotary die soft gelatin capsule machine as described in The Theory and Practice of Industrial Pharmacy, ed. Lachman, et al., 2nd Ed., Pt. II, 404-420, Lea & Febiger, 1976. Additional methods include using a plate process (see The Theory and Practice of Industrial Pharmacy, ed. Lachman, et al., 2nd Ed., Pt. II, 405, Lea & Febiger, 1976), as well as Globex type seamless capsule machines, which makes large microcapsules (see U.S. Pat. No. 5,254,294), non-standard rotary die machines, which uses extrusion technology to make gel ribbons (see U.S. Pat. Nos. 6,183,845 and 6,340,473), and other methods for making capsules which use high frequency, ultrasonic, or induction welding to seal the capsules (see U.S. Pat. No. 6,352,719). The above-listed U.S. patents and book are hereby incorporated by reference.

As used herein, the phrase “liquid hard-shell” refers to a hard capsule encapsulating a liquid or semi-solid formulation. Hard capsules can be single unit dosage forms and may comprise a cap and a body, which can be manufactured separately, and which can be supplied empty for filling with the liquid or semi-solid composition. In some embodiments, hard capsules are made from a polymer such as gelatin. An additional component can be water, which acts as a plasticizer. Another hard capsule may be manufactured from hydroxypropylmethyl cellulose (HPMC). Liquid-fill hard capsule can be filled on a filling machine, such as, for example, a high-speed filling machine.

In one example, disclosed herein is a method of prepare the filled hard capsule. Empty capsules are supplied to the filling machine in a prelocked condition, wherein the capsule body has a cap which is loosely attached thereto. A series of rings or protrusions are provided in the mating surfaces of the cap or body. These rings are configured to enable the cap to be loosely attached to the body so that the cap and body are held together during storage but would enable the cap to be removed prior to filling of the capsule. Once the capsule has been filled, the cap can be replaced and be forced beyond the prelocked position into a fully locked position. Alternatively, other types of capsule filling machines can be used to accept separate supplies of capsule bodies and caps.

The capsules may be closed at high speed after filling with the formulated composition. During closure of the capsule, the cap is fitted over the body and the body is pushed up until it locks on the cap. The cap can be close fitting and can be approximately half the length of the body, so the cap can travel for a considerable distance down the capsule body before locking. This may have the effect of a piston in trapping and pressurizing the capsule. The excess gas can escape through the gap between the cap and the body, and vents may be provided in this region so as to facilitate the escape of excess pressure. Alternatively, the capsule may utilize a particularly tight locking mechanism rather than vents

In an embodiment, the capsule is banded by applying a band of polymer solution around the junction between the cap and body. The polymer solution can be a solution of the same polymer as the capsule cap and/or body in a solvent therefor. Banding can provide a smooth capsule surface for coating, which may prevent movement between the cap and body of the capsule.

When preparing the filled capsule that is filled with the composition comprising an avermectin, it is preferred that the composition is in a liquid form at least during the encapsulation process. In an embodiment, the final capsule contains the composition in the liquid form. In an embodiment, the final capsule contains the composition is semi-solid form at room temperature.

### Method of Administration

The administration of the pharmaceutical composition comprising the avermectin can be carried out via oral, nasal, intraocular, intravenous, intramuscular, subcutaneous, transdermal, subdermal, sublingual or rectal route of administration.

In embodiments, the route of administration is oral and the pharmaceutical composition is provided in the form of capsules, such as soft elastic or hard gelatin capsules.

In an embodiment, the route of administration is nasal. The composition can be a solution, an aerosol, a liquid suspension, or a liquid dispersion, in the form of a nasal spray, a nasal douche, an inhaler, a nasal drop, and/or a diffuser.

In embodiments, the route of administration is dermal including but not limited to topical, subcutaneous, subdermal, transdermal, intradermal or dermal patch.

### Methods of Treatment and Prevention

Provided herein are methods of treating or preventing a neurological or other disorders by administering to a subject in need thereof a therapeutically or a prophylactically effective amount of a composition disclosed herein.

A “therapeutically effective amount” of a composition comprising an (i) a compound of any one of Formulas I-VII, (ii) one or more of a compound of any one of Formulas I-VII, or (iii) ivermectin comprising a compound of Formula VI and a compound of Formula VII is an amount sufficient to confer a therapeutic benefit in a patient after administration for example to improve in the subject one or more symptoms of the disease. The “therapeutically effective amount” may result in a desired beneficial change of physiology in the subject or to cause an improvement in a clinically significant condition in the subject, for example, by delaying, reducing, minimizing or mitigating one or more symptoms associated with the disease or disorder. Generally, a therapeutically effective amount is also one in which any toxic or detrimental effects of a composition are outweighed by the therapeutically beneficial effects The effective amount may vary depending on the species, age, weight, sex, health of the subject and the nature or severity of the disease. Depending on the mode of administration, the effective amount may vary as well. In some cases, multiple doses of the composition are administered to achieve the effective amount for the therapeutic benefit intended. In some cases, the therapeutic amount may be used for treating refractory or resistant disorders, and in combination therapies. For example, the effective amount may be administered simultaneously, sequentially, and in the same or different dosage form as an adjunct therapy.

As used herein, the terms “treating”, “treat”, “treatment” refer to reducing, relieving, ameliorating, or alleviating at least one of the symptoms of the disease or disorder. The term includes, for example, administering a formulation as provided herein prevent the onset of spasticity, to reduce or alleviate its severity, and/or to prevent its reoccurrence.

As used herein, the terms “prevent”, “prevention”, and the like refer to acting prior to overt disease or disorder onset, to prevent the disease or disorder from developing or to minimize the extent of the disease or disorder, or slow its course of development.

A “prophylactically effective amount” of a composition comprising an (i) a compound of any one of Formulas I-VII, (ii) one or more of a compound of any one of Formulas I-VII, or (iii) ivermectin comprising a compound of Formula VI and a compound of Formula VII refers to an amount of a composition required to achieve a desired prophylactic result. In an embodiment, the prophylactically effective amount is less than the therapeutically effective amount, as a prophylactic dose is used in subjects prior to or at an earlier stage of disease.

A subject may be a mammal, including, but not limited to, a human or non-human mammal. The mammal may be a commercially farmed animal (such as a horse, a cow, a sheep or a pig), a laboratory animal (such as a mouse or a rat), or a pet (such as a cat, a dog, a rabbit or a guinea pig). The subject is preferably a human. The subject may be male or female. Individuals and patients are also subjects herein.

### Neurological Disorders

The disclosure provides a method of treating or preventing a neurological disorder by administering to a subject in need thereof a therapeutically or a prophylactically effective amount of (i) a compound of any one of Formulas I-VII, (ii) one or more of a compound of any one of Formulas I-VII, or (iii) ivermectin comprising a compound of Formula VI and a compound of Formula VII.

Ivermectin has been shown to interact with the purinergic P2X4 receptors, 7-aminobutyric acid A (GABA_{A}) receptor, glycine receptor (GlyR), and neuronal alpha-7-nicotinic receptor in humans; the EC50 for each of the above interactions is about 0.25 μM, 0.92 μM, 1.2 μM, and 30 μM, respectively. These channels play critical roles in epilepsy. As such, ivermectin can be used as an anticonvulsant agent. The anticonvulsant effect of ivermectin can be achieved via a collective effect from any combinations of the interactions with these four receptors described above.

Ivermectin can be mostly lipophilic, which can be an advantage to use in the central nervous system as lipophilic agents may readily cross the blood-brain barrier (BBB). The blood-brain barrier can be an impediment to the entry of many therapeutic drugs into the brain. However, ivermectin may have a limited half-life in the brain after crossing the BBB. The reason may be that ivermectin can be a substrate of P-glycoprotein. Specifically, multidrug resistance protein 1 (MDR1), also called P-glycoprotein or ATP-binding cassette subfamily B member 1 (ABCB1), is an efflux transport system that selectively transports substrates from the interstitial fluid (ISF) to the blood. P-Glycoprotein can restrict the entry of materials from the blood into the brain parenchyma. P-Glycoprotein, an ATP-dependent drug transport protein, can be found in the apical membranes of a number of epithelial cell types in the body, including the blood luminal membrane of the brain capillary endothelial cells. The brain capillary endothelial cells can make up the blood-brain barrier. Since P-glycoprotein can actively transport a huge variety of hydrophobic amphipathic drugs out of the cell, it can be responsible for pumping out hydrophobic drugs from the brain, by performing active back-transport of these drugs to the blood. Therefore, after crossing the BBB, ivermectin can get pumped out of the brain cells and back into the blood. Accordingly, provided herein are compositions that address the need for formulations with improved pharmacokinetic properties, including, but not limited to bioavailability.

Accordingly, in one aspect, the disclosure provides a method for treating or preventing neurological disorders featuring a seizure disorder including but not limited to epilepsy, treatment-resistant epilepsy, Gravet syndrome, Lenox Gastaut syndrome, spinal cord injury, childhood absence 5 (ECAS), epileptic encephalopathy (EE), early infantile epileptic encephalopathy 43 (EIEE43), Angelman syndrome, injury to brain, stroke, addictive behavior, subarachnoid hemorrhage, anoxic encephalopathy, infectious or metabolic encephalopathy, hemorrhagic, embolic/athersclerotic cerebrovascular accidents, and other seizure indications.

In another aspect the disclosure provides a method for treating or preventing epilepsy. In some embodiments, the epilepsy is refractory or treatment-resistant epilepsy. In embodiments, this method comprises administering a composition comprising (i) a compound of any one of Formulas I-VII, (ii) one or more of a compound of any one of Formulas I-VII, or (iii) ivermectin comprising a compound of Formula VI and a compound of Formula VII with one or more adjunct therapies including up to four anti-epileptic drugs (AEDs), administered simultaneously, sequentially, and in the same or different dosage form as a therapeutically effective or a prophylactically effective amount of a composition, or compositions, comprising one or more avermectins, and particularly ivermectin or an ivermectin derivative. The AEDs can include drugs such as lorazepam, cenobamate, brivaracetam, striripentol, acetazolamide, phenytoin, sodium valproate, buccal midazolam, felbarnate, clobazam, permpanel, tiagabine, rufinamide, levetiracetam, larnotrigine, pregabalin, primidone, gabapentin, nitrazepam, phenobarbital, clonazepam, vigabatrin, carbamazepine, topiramate, oxcarbazepine, rectal diazepam, lacosamide, ethosuximide, eslicarbazepine acetate, and/or zonisamide. Non-pharmaceutical anti-epileptic therapies may also be administered concomitantly, including implantable devices such as vagal nerve stimulators, responsive neurostimulators, and deep brain stimulators.

In another aspect, the disclosure provides a method for treating or preventing neurological disorders associated with muscle movement disorders including but not limited to multiple sclerosis, essential tremor, multiple forms of spasticity including spasticity associated with spinal cord injury, spasticity associated with other diseases including parasitic paresis, spasticity associated with cerebral palsy, incontinence after spinal cord injury, dystonia, lateral sclerosis, myotonic dystrophy, congenital (hereditary) muscular dystrophies, e.g. Duchenne's and Becker's, Rett syndrome, Prader-Willi syndrome and any orphan motor neuron diseases.

In one aspect, the disclosure provides a method for treating or preventing neurological disorders caused by GABAergic dysfunction including but not limited to Alzheimer's, Parkinson's disease, Schizophrenia, Autism, autism spectrum disorder, global developmental delay, decreased fine and gross motor control, or attention deficit hyperactivity disorder (ADHD).

In one aspect, the disclosure provides a method for treating or preventing neurological disorders caused by glycinergic dysfunction including but not limited to stiff person syndrome or startle disease.

In one aspect, the disclosure provides a method to treating or preventing neurological disorders caused by an infection including but not limited to meningitis, meningoencephalitis, encephalitis, mycobacterium infection, Zika infection, cerebral malaria, or abscesses in the central nervous system.

In one aspect, the disclosure provides a method to treating or preventing neurological disorders caused by an injury.

### Spinal Spasticity

In one aspect, the disclosure provides a method of treating or preventing spinal spasticity by administering to a subject in need thereof a therapeutically or a prophylactically effective amount of (i) a compound of any one of Formulas I-VII, (ii) one or more of a compound of any one of Formulas I-VII, or (iii) ivermectin comprising a compound of Formula VI and a compound of Formula VII.

Spasticity is a disorder in which certain muscles are continuously contracted. Spasticity may produce uncontrollable, painful spasms of the legs or other extremities. Spasticity can also interfere with movement and speech. Untreated spasticity can lead to frozen or immobilized joints and pressure sores. It can prevent recovery of proper motor behavior after injury or disease. Further, spasticity may cause pain, fatigue and other problems. As such, spasticity can become a barrier to a person's daily activities, walking, sitting, positioning and sleep.

The degree of spasticity may vary from mild muscle stiffness to severe and uncontrollable muscle spasms. Spasticity can be very painful and, depending on the affected muscles, can result in an uncoordinated gait, stiff or deformed posture, and shortening of the range of limb movement. It can cause permanent muscle shortening and problems around the joints against which the two spastic muscles are supposed to move (contracture). It can be a permanent feature or brought on by a variety of factors such as fatigue, heat, or infection.

A major component of spasticity is exaggerated reflexes or hyper-reflexia. One measure to quantify hyper-reflexia is the electrical analogue of the classic tendon jerk reflex, referred to as the Hoffman or H-reflex. The H-reflex is a compound electromyographic (EMG) response elicited by the synaptic activation of motoneurons by muscle afferents following stimulation of muscle nerves.

Spasticity may occur in association with spinal cord injury; damage to the brain because of lack of oxygen, stroke, or head injury; amyotrophic lateral sclerosis (Lou Gehrig's disease); phenylketonuria; metabolic diseases such as adrenoleukodystrophy; cerebral palsy; Stiff-man Syndrome; and multiple sclerosis. In multiple sclerosis, it may affect the legs, although it can affect almost any muscle pair in the body. In multiple sclerosis, spasticity is usually caused by damage to the nerves (neurons) that control muscles or those that collect sensory information back from them. Reflexive spasms which are generated by the spinal cord are not inhibited by the brain, as normal, and increased muscle tone results. The lesions responsible are usually in the cerebellum or the white matter tracts that connect it to the peripheral motor (efferent) and sensory (afferent) nerves.

Current drugs available for the management of spasticity include baclofen, which is a GABA_{B} receptor agonist. Baclofen acts as an agonist to potentiate inhibition mediated by presynaptic GABA_{B} receptors. The rationale for the use of baclofen for the treatment of spasticity was due to the assumption that excessive reflexes were induced in spinal cord injury by the elimination of presynaptic inhibition from nerve fibers descending from the brain. spinal cord injury has been thought to lesion these descending fibers, rendering the spinal cord hyper-reactive. Baclofen is thought to restore some of that inhibition. However, baclofen taken orally unfortunately increases inhibition throughout the brain, leading to sleepiness and weakness. In order to avoid such unpleasant side effects (which reduce the ability of patients to concentrate), baclofen pumps were designed to inject the drug directly into the spinal fluid, usually in spinal cord injury victims and cerebral palsy patients. These pumps avoid the soporific effects of oral administration but are dangerous, have a limited lifetime (requiring repeated surgical implants) and are very expensive.

In an embodiment, the treatment reduces hyper-reflexia in the human subject. In an embodiment, a reduction in hyper-reflexia is used to evaluate the reduction of spasticity. As used herein, the term “reduce hyper-reflexia” refers to reducing the amplitude of the H-reflex measured at 1, 5, or 10 Hz as normalized to amplitude measured at 0.2 Hz stimulation. In other words, it refers to restoring low frequency-dependent depression of the H-reflex to closer to control levels. More preferably the H-reflex amplitude is reduced at 5 or 10 Hz, most preferably at 5 Hz.

In some embodiments, the formulations and methods disclosed herein may be effective in reducing the degree of stiffness of spasticity.

In one aspect, the disclosure provides a method for treating or preventing spinal spasticity secondary to a disease including, but not limited to, spinal cord injury, stroke, brain injury, amyotrophic lateral sclerosis, phenylketonuria, adrenoleukodystrophy, cerebral palsy, Stiff-man Syndrome, or multiple sclerosis.

In an embodiment, the spasticity is secondary to a spinal cord injury, and wherein the method further comprises beginning administering to the human subject a dose of the formulation after the spinal cord injury and before the spasticity develops.

In an embodiment, the spasticity is secondary to multiple sclerosis, wherein the method further comprises beginning administering to the human subject a dose of the formulation after a diagnosis of the multiple sclerosis and before the spasticity. In an embodiment, administering the formulation is effective to increase connexin 36 levels in spinal cord neuron, wherein administering the formulation is effective to increase gap junctions in neurons in the spinal cord or increase electrical coupling between neurons in the spinal cord.

The subjects receiving a treatment described herein may experience as a result of the therapy a reduction of spasticity.

The level of Protein connexin36 (Cx36) can be used to evaluate the reduction of spasticity. Cx36 forms gap junctions between neurons, which are called electrical synapses, enabling adjacent neurons to communicate directly. Passive exercise, which also reduces spasticity, increases expression of the nerve gap junction protein connexin 36, which would increase gap junctions and increase electrical communication between neurons.

Another means of evaluating the effectiveness of a dose is by evaluating muscle tone. In spasticity there is a disruption in the normal behavior of the stretch reflex that causes muscles, particularly the flexors, to be extremely resistive to passive stretch, that is, to be high in tone. As a result, motor control is severely impaired, and stiffness or tightness of the muscles may interfere with gait, movement, and speech. Hence, assessing tone—the degree of resistance to stretch from an external source—is an important means by which one can evaluate the degree of spasticity that a patient has and the effectiveness of intervention.

Another accepted clinical measures of tone in spasticity is the Ashworth scale and the Modified Ashworth Scale. A clinician moves a patient's limbs about their joints and then assigns a grade to each limb corresponding to how much resistance the clinician feels.

Other means to evaluate the effectiveness of a dose include biomechanical studies, such as those performed by a pendulum test; electrophysiologic studies, such as by electromyography (such as dynamic multichannel electromyography with gait studies) or Hoffman reflex studies (measuring deep tendon reflexes); and functional measurements, such as those given by the Barthel Index, Functional Independence Measure, and Fugl-Meyer Assessment of Sensorimotor Impairment (Fugl-Meyer scale).

In an embodiment, provided is a method of treating or preventing spinal spasticity by administering to a subject in need thereof a composition disclosed herein, the method further comprising administering to the human subject a second therapeutic agent. In some embodiments, the second agent is baclofen, benzodiazepines, diazepam, clonazepam, dantrolene, or tizanidine.

### Pain

In one aspect, the disclosure provides a method of treating or preventing pain by administering to a subject in need thereof a therapeutically effective or a prophylactically effective amount of (i) a compound of any one of Formulas I-VII, (ii) one or more of a compound of any one of Formulas I-VII, or (iii) ivermectin comprising a compound of Formula VI and a compound of Formula VII.

In some embodiments, the pain is pain that is associated with a disease including but not limited to post-traumatic stress disorder (PTSD), gastroesophageal reflex disease (GERD), addiction (e.g., alcohol, drugs), anxiety, depression, memory loss, dementia, sleep apnea, stroke, urinary incontinence, narcolepsy, essential tremor, movement disorder, atrial fibrillation, cancer (e.g., brain tumors), Parkinson's disease, or Alzheimer's disease. Other non-limiting examples of neurological diseases or disorders that can be treated or prevented by the compositions and methods herein include: Abulia, Agraphia, Alcoholism, Alexia, Aneurysm, Amaurosis fugax, Amnesia, Amyotrophic lateral sclerosis (ALS), Angelman syndrome, Aphasia, Apraxia, Arachnoiditis, Arnold-Chiari malformation, Asperger syndrome, Ataxia, Ataxia-telangiectasia, Attention deficit hyperactivity disorder, Auditory processing disorder, Autism spectrum, Bipolar disorder, Bell's palsy, Brachial plexus injury, Brain damage, Brain injury, Brain tumor, Canavan disease, Capgras delusion, Carpal tunnel syndrome, Causalgia, Central pain syndrome, Central pontine myelinolysis, Centronuclear myopathy, Cephalic disorder, Cerebral aneurysm, Cerebral arteriosclerosis, Cerebral atrophy, Cerebral autosomal dominant arteriopathy with subcortical infarcts and leukoencephalopathy (CADASIL), Cerebral gigantism, Cerebral palsy, Cerebral vasculitis, Cervical spinal stenosis, Charcot-Marie-Tooth disease, Chiari malformation, Chorea, Chronic fatigue syndrome, Chronic inflammatory demyelinating polyneuropathy (CIDP), Chronic pain, Coffin-Lowry syndrome, Coma, Complex regional pain syndrome, Compression neuropathy, Congenital facial diplegia, Corticobasal degeneration, Cranial arteritis, Craniosynostosis, Creutzfeldt-Jakob disease, Cumulative trauma disorders, Cushing's syndrome, Cyclothymic disorder, Cytomegalic inclusion body disease (CIBD), Cytomegalovirus Infection, Dandy-Walker syndrome, Dawson disease, De Morsier's syndrome, Dejerine-Klumpke palsy, Dejerine-Sottas disease, Delayed sleep phase syndrome, Dementia, Dermatomyositis, Developmental coordination disorder, Diabetic neuropathy, Diffuse sclerosis, Diplopia, Down syndrome, Dravet syndrome, Duchenne muscular dystrophy, Dysarthria, Dysautonomia, Dyscalculia, Dysgraphia, Dyskinesia, Dyslexia, Dystonia, Empty sella syndrome, Encephalitis, Encephalocele, Encephalotrigeminal angiomatosis, Encopresis, Enuresis, Epilepsy, Epilepsy-intellectual disability in females, Erb's palsy, Erythromelalgia, Exploding head syndrome, Fabry's disease, Fahr's syndrome, Fainting, Familial spastic paralysis, Febrile seizures, Fisher syndrome, Friedreich's ataxia, Fibromyalgia, Foville's syndrome, Fetal alcohol syndrome, Fragile X syndrome, Fragile X-associated tremor/ataxia syndrome (FXTAS), Gaucher's disease, Generalized epilepsy with febrile seizures plus, Gerstmann's syndrome, Giant cell arteritis, Giant cell inclusion disease, Globoid Cell Leukodystrophy, Gray matter heterotopia, Guillain-Barre syndrome, Generalized anxiety disorder, HTLV-1 associated myelopathy, Hallervorden-Spatz disease, Head injury, Headache, Hemifacial Spasm, Hereditary Spastic Paraplegia, Heredopathia atactica polyneuritiformis, Herpes zoster oticus, Herpes zoster, Hirayama syndrome, Hirschsprung's disease, Holmes-Adie syndrome, Holoprosencephaly, Huntington's disease, Hydranencephaly, Hydrocephalus, Hypercortisolism, Hypoxia, Immune-Mediated encephalomyelitis, Inclusion body myositis, Incontinentia pigmenti, Infantile Refsum disease, Infantile spasms, Inflammatory myopathy, Intracranial cyst, Intracranial hypertension, Isodicentric 15, Joubert syndrome, Karak syndrome, Kearns-Sayre syndrome, Kinsbourne syndrome, Kleine-Levin Syndrome, Klippel Feil syndrome, Krabbe disease, Lafora disease, Lambert-Eaton myasthenic syndrome, Landau-Kleffner syndrome, Lateral medullary (Wallenberg) syndrome, Learning disabilities, Leigh's disease, Lennox-Gastaut syndrome, Lesch-Nyhan syndrome, Leukodystrophy, Leukoencephalopathy with vanishing white matter, Lewy body dementia, Lissencephaly, Locked-In syndrome, Lumbar disc disease, Lumbar spinal stenosis, Lyme disease—Neurological Sequelae, Machado-Joseph disease (Spinocerebellar ataxia type 3), Macrencephaly, Macropsia, Mal de debarquement, Megalencephalic leukoencephalopathy with subcortical cysts, Megalencephaly, Melkersson-Rosenthal syndrome, Menieres disease, Meningitis, Menkes disease, Metachromatic leukodystrophy, Microcephaly, Micropsia, Migraine, Miller Fisher syndrome, Mini-stroke (transient ischemic attack), Misophonia, Mitochondrial myopathy, Mobius syndrome, Monomelic amyotrophy, Motor skills disorder, Moyamoya disease, Mucopolysaccharidoses, Multi-infarct dementia, Multifocal motor neuropathy, Multiple sclerosis, Multiple system atrophy, Muscular dystrophy, Myalgic encephalomyelitis, Myasthenia gravis, Myelinoclastic diffuse sclerosis, Myoclonic Encephalopathy of infants, Myoclonus, Myopathy, Myotubular myopathy, Myotonia congenita, Narcolepsy, Neuro-Beçget's disease, Neurofibromatosis, Neuroleptic malignant syndrome, Neurological manifestations of AIDS, Neurological sequelae of lupus, Neuromyotonia, Neuronal ceroid lipofuscinosis, Neuronal migration disorders, Neuropathy, Neurosis, Niemann-Pick disease, Non-24-hour sleep-wake disorder, Nonverbal learning disorder, O'Sullivan-McLeod syndrome, Occipital Neuralgia, Occult Spinal Dysraphism Sequence, Ohtahara syndrome, Olivopontocerebellar atrophy, Opsoclonus myoclonus syndrome, Optic neuritis, Orthostatic Hypotension, Otosclerosis, Overuse syndrome, Palinopsia, Paresthesia, Parkinson's disease, Paramyotonia Congenita, Paraneoplastic diseases, Paroxysmal attacks, Parry-Romberg syndrome, PANDAS, Pelizaeus-Merzbacher disease, Periodic Paralyses, Peripheral neuropathy, Pervasive developmental disorders, Photic sneeze reflex, Phytanic acid storage disease, Pick's disease, Pinched nerve, Pituitary tumors, PMG, Polyneuropathy, Polio, Polymicrogyria, Polymyositis, Porencephaly, Post-Polio syndrome, Postherpetic Neuralgia (PHN), Postural Hypotension, Prader-Willi syndrome, Primary Lateral Sclerosis, Prion diseases, Progressive hemifacial atrophy, Progressive multifocal leukoencephalopathy, Progressive Supranuclear Palsy, Prosopagnosia, Pseudotumor cerebri, Quadrantanopia, Quadriplegia, Rabies, Radiculopathy, Ramsay Hunt syndrome type I, Ramsay Hunt syndrome type II, Ramsay Hunt syndrome type III, Rasmussen encephalitis, Reflex neurovascular dystrophy, Refsum disease, REM sleep behavior disorder, Repetitive stress injury, Restless legs syndrome, Retrovirus-associated myelopathy, Rett syndrome, Reye's syndrome, Rhythmic Movement Disorder, Romberg syndrome, Saint Vitus dance, Sandhoff disease, Schilder's disease, Schizencephaly, Sensory processing disorder, Septo-optic dysplasia, Shaken baby syndrome, Shingles, Shy-Drager syndrome, Sjögren's syndrome, Sleep apnea, Sleeping sickness, Snatiation, Sotos syndrome, Spasticity, Spina bifida, Spinal cord injury, Spinal cord tumors, Spinal muscular atrophy, Spinal and bulbar muscular atrophy, Spinocerebellar ataxia, Split-brain, Steele-Richardson-Olszewski syndrome, Stiff-person syndrome, Stroke, Sturge-Weber syndrome, Stuttering, Subacute sclerosing panencephalitis, Subcortical arteriosclerotic encephalopathy, Superficial siderosis, Sydenham's chorea, Syncope, Synesthesia, Syringomyelia, Tarsal tunnel syndrome, Tardive dyskinesia, Tardive dysphrenia, Tarlov cyst, Tay-Sachs disease, Temporal arteritis, Temporal lobe epilepsy, Tetanus, Tethered spinal cord syndrome, Thomsen disease, Thoracic outlet syndrome, Tic Douloureux, Todd's paralysis, Tourette syndrome, Toxic encephalopathy, Transient ischemic attack, Transmissible spongiform encephalopathies, Transverse myelitis, Traumatic brain injury, Tremor, Trichotillomania, Trigeminal neuralgia, Tropical spastic paraparesis, Trypanosomiasis, Tuberous sclerosis, Unverricht-Lundborg disease, Von Hippel-Lindau disease (VHL), Viliuisk Encephalomyelitis (VE), Wallenberg's syndrome, West syndrome, Whiplash, Williams syndrome, Wilson's disease, or Zellweger syndrome.

In another aspect, the disclosure provides a method for treating or preventing pain in a subject. “Pain” refers to an uncomfortable feeling and/or an unpleasant sensation in the body of a subject. Feelings of pain can range from mild and occasional to severe and constant. Pain can be classified as acute pain or chronic pain. Pain can be nociceptive pain (i.e., pain caused by tissue damage), neuropathic pain or psychogenic pain. In some cases, the pain is caused by or associated with a disease (e.g., cancer, arthritis, diabetes). In other cases, the pain is caused by injury (e.g., sports injury, trauma). Non-limiting examples of pain that are amenable to treatment or prevention with the compositions and methods herein include: neuropathic pain including peripheral neuropathy, diabetic neuropathy, post herpetic neuralgia, trigeminal neuralgia, back pain, neuropathy associated with cancer, neuropathy associated with HIV/AIDS, phantom limb pain, carpal tunnel syndrome, central post-stroke pain, pain associated with chronic alcoholism, hypothyroidism, uremia, pain associated with multiple sclerosis, pain associated with spinal cord injury, pain associated with Parkinson's disease, epilepsy, osteoarthritic pain, rheumatoid arthritis pain, visceral pain, and pain associated with vitamin deficiency; and nociceptive pain including pain associated with central nervous system trauma, strains/sprains, and burns; myocardial infarction, acute pancreatitis, post-operative pain, posttraumatic pain, renal colic, pain associated with cancer, pain associated with fibromyalgia, pain associated with carpal tunnel syndrome, and back pain.

Illustrative examples of pain that are amenable to treatment or prevention with the formulations, and methods include but are not limited to acute pain, chronic pain, neuropathic pain, nociceptive pain, allodynia, inflammatory pain, inflammatory hyperalgesia, neuropathies, neuralgia, diabetic neuropathy, human immunodeficiency virus-related neuropathy, nerve injury, rheumatoid arthritis pain, osteoarthritic pain, burns, back pain, eye pain, visceral pain, cancer pain (e.g., bone cancer pain), dental pain, headache, migraine, carpal tunnel syndrome, fibromyalgia, neuritis, sciatica, pelvic hypersensitivity, pelvic pain, post herpetic neuralgia, post-operative pain, post stroke pain, and menstrual pain.

Pain can be classified as acute or chronic. As used herein, the term “acute pain” refers to pain that begins suddenly and is usually sharp in quality. Acute pain might be mild and last just a moment, or it might be severe and last for weeks or months. In most cases, acute pain does not last longer than three months, and it disappears when the underlying cause of pain has been treated or has healed. Unrelieved acute pain, however, may lead to chronic pain. As used herein, the term “chronic pain” refers to ongoing or recurrent pain, lasting beyond the usual course of acute illness or injury or lasting for more than three to six months, and which adversely affects the individual's well-being. In an embodiment, the term “chronic pain” refers to pain that continues when it should not. Chronic pain can be nociceptive pain or neuropathic pain.

In some embodiments, the formulations and methods may be effective in reducing acute pain. In some embodiments, the formulations and methods may be effective in reducing chronic pain.

Clinical pain is present when discomfort and abnormal sensitivity feature among the patient's symptoms. Individuals can present with various pain symptoms. Such symptoms may include: 1) spontaneous pain which may be dull, burning, or stabbing; 2) exaggerated pain responses to noxious stimuli (hyperalgesia); and 3) pain produced by normally innocuous stimuli. Pain can also be divided into a number of different subtypes according to differing pathophysiology, including nociceptive pain, inflammatory pain, and neuropathic pain.

In some embodiments, the formulations and methods may be effective in reducing nociceptive pain. In some embodiments, the formulations and methods may be effective in reducing inflammatory pain. In some embodiments, the compositions and methods may be effective in reducing neuropathic pain.

Nociceptive pain is induced by tissue injury or by intense stimuli with the potential to cause injury. Nonlimiting examples of pain include pain from central nervous system trauma, strains/sprains, burns, myocardial infarction and acute pancreatitis, post-operative pain (pain following any type of surgical procedure), posttraumatic pain, renal colic, cancer pain and back pain. Cancer pain may be chronic pain such as tumor related pain (e.g., bone pain, headache, facial pain or visceral pain) or pain associated with cancer therapy (e.g., post chemotherapy syndrome, chronic postsurgical pain syndrome or post radiation syndrome). Cancer pain may also occur in response to chemotherapy, immunotherapy, hormonal therapy or radiotherapy. Back pain may be due to herniated or ruptured intervertebral discs or abnormalities of the lumber facet joints, sacroiliac joints, paraspinal muscles or the posterior longitudinal ligament.

Neuropathic pain may be pain initiated or caused by a primary lesion or dysfunction in the nervous system. Etiologies of neuropathic pain include, e.g., peripheral neuropathy, diabetic neuropathy, post herpetic neuralgia, trigeminal neuralgia, back pain, cancer neuropathy, HIV neuropathy, phantom limb pain, carpal tunnel syndrome, central post-stroke pain and pain associated with chronic alcoholism, hypothyroidism, uremia, multiple sclerosis, spinal cord injury, Parkinson's disease, epilepsy, and vitamin deficiency.

Neuropathic pain can be related to a pain disorder, a term referring to a disease, disorder or condition associated with or caused by pain. Illustrative examples of pain disorders include arthritis, allodynia, a typical trigeminal neuralgia, trigeminal neuralgia, somatoform disorder, hypoesthesis, hypealgesia, neuralgia, neuritis, neurogenic pain, analgesia, anesthesia dolorosa, causlagia, sciatic nerve pain disorder, degenerative joint disorder, fibromyalgia, visceral disease, chronic pain disorders, migraine/headache pain, chronic fatigue syndrome, complex regional pain syndrome, neurodystrophy, plantar fasciitis or pain associated with cancer.

The inflammatory process is a complex series of biochemical and cellular events, activated in response to tissue injury or the presence of foreign substances, which results in swelling and pain. Arthritic pain is a common inflammatory pain.

Other types of pain that are amenable to treatment or prevention with the formulations, and methods may include but are not limited to pain resulting from musculoskeletal disorders, including myalgia, fibromyalgia, spondylitis, sero-negative (non-rheumatoid) arthropathies, non-articular rheumatism, dystrophinopathy, glycogenolysis, polymyositis and pyomyositis; heart and vascular pain, including pain caused by angina, myocardial infarction, mitral stenosis, pericarditis, Raynaud's phenomenon, scleroderma and skeletal muscle ischemia; head pain, such as migraine (including migraine with aura and migraine without aura), cluster headache, tension-type headache mixed headache and headache associated with vascular disorders; and orofacial pain, including dental pain, otic pain, burning mouth syndrome, and temporomandibular myofascial pain.

The effective amount of the compositions and methods to reduce the amount of pain experienced by a human subject can be determined using a variety of pain scales. Patient self-reporting can be used to assess whether pain is reduced; see, e.g., Katz and Melzack (1999) Surg. Clin. North Am. 79:231. Alternatively, an observational pain scale can be used. The LANSS Pain Scale can be used to assess whether pain is reduced; see, e.g., Bennett (2001) Pain 92:147. A visual analog pain scale can be used; see, e.g., Schmader (2002) Clin. J. Pain 18:350. The Likert pain scale can be used; e.g., where 0 is no pain, 5 is moderate pain, and 10 is the worst pain possible. Self-reported pain scales for children include, e.g., Faces Pain Scale; Wong-Baker FACES Pain Rating Scale; and Colored Analog Scale. Self-report pain scales for adults include, e.g., Visual Analog Scale; Verbal Numerical Rating Scale; Verbal Descriptor Scale; and Brief Pain Inventory. Pain measurement scales include, e.g., Alder Hey Triage Pain Score (Stewart et al. (2004) Arch. Dis. Child. 89:625); Behavioral Pain Scale (Payen et al. (2001) Critical Care Medicine 29:2258); Brief Pain Inventory (Cleeland and Ryan (1994) Ann. Acad. Med. Singapore 23: 129); Checklist of Nonverbal Pain Indicators (Feldt (2000) Pain Manag. Nurs. 1: 13); Critical-Care Pain Observation Tool (Gelinas et al. (2006) Am. J. Crit. Care 15:420); COMFORT scale (Ambuel et al. (1992) J. Pediatric Psychol. 17:95); Dallas Pain Questionnaire (Ozguler et al. (2002) Spine 27:1783); Dolorimeter Pain Index (Hardy et al. (1952) Pain Sensations and Reactions Baltimore: The Williams & Wilkins Co.); Faces Pain Scale-Revised (Hicks et al. (2001) Pain 93:173); Face Legs Activity Cry Consolability Scale; McGill Pain Questionnaire (Melzack (1975) Pain 1:277); Descriptor Differential Scale (Gracely and Kwilosz (1988) Pain 35:279); Numerical 1 1 point Box (Jensen et al. (1989) Clin. J. Pain 5: 153); Numeric Rating Scale (Hartrick et al. (2003) Pain Pract. 3:310); Wong-Baker FACES Pain Rating Scale; and Visual Analog Scale (Huskisson (1982) J. Rheumatol. 9:768).

### Dosing Regimens

Provided herein are methods of treating or preventing a neurological or other disorder including, but not limited to treatment resistant-epilepsy, by administering to a subject in need thereof a therapeutically or a prophylactically effective amount of a composition, the method comprising administering one of the compositions disclosed herein at a dose of about 10 mg to about 120 mg of (i) a compound of any one of Formulas I-VII, (ii) one or more of a compound of any one of Formulas I-VII, or (iii) ivermectin comprising a compound of Formula VI and a compound of Formula VII. In some embodiments, the method of treatment or prevention includes administering a composition disclosed herein at a dose of about 10 mg to about 80 mg of (i) a compound of any one of Formulas I-VII, (ii) one or more of a compound of any one of Formulas I-VII, or (iii) ivermectin comprising a compound of Formula VI and a compound of Formula VII. In some embodiments, the method of treatment or prevention includes administering a composition disclosed herein at a dose of about 10 mg to about 60 mg of (i) a compound of any one of Formulas I-VII, (ii) one or more of a compound of any one of Formulas I-VII, or (iii) ivermectin comprising a compound of Formula VI and a compound of Formula VII. In some embodiments, the method of treatment or prevention includes administering a composition disclosed herein at a dose of about 20 mg to about 40 mg of (i) a compound of any one of Formulas I-VII, (ii) one or more of a compound of any one of Formulas I-VII, or (iii) ivermectin comprising a compound of Formula VI and a compound of Formula VII. In some embodiments, the method of treatment or prevention includes administering a composition disclosed herein at a dose of about 1 mg, about 3 mg, about 5 mg, about 10 mg, about 20 mg, about 40 mg, about 60 mg, about 80 mg, or about 120 mg of (i) a compound of any one of Formulas I-VII, (ii) one or more of a compound of any one of Formulas I-VII, or (iii) ivermectin comprising a compound of Formula VI and a compound of Formula VII. In preferred embodiments, the composition comprises ivermectin comprising a compound of Formula VI and a compound of Formula VII.

The dosage regimens for the therapy may be adjusted to provide the optimum desired response (e.g., a therapeutic or prophylactic response). For example, a single dose may be administered, several divided doses may be administered over time or the dose may be proportionally reduced or increased depending on the subject's responsiveness to the therapy. In some embodiments, the composition is administered twice per day (BD), once per day (QD), once every other day, once every three days. In some embodiments, the dosage schedule is once a day, twice a day, every other day or once every three days for about 14 days, for about 30 days, for about 60 days, for about 84 days, for about 90 days, or continuously. In some embodiments, the dosage schedule is daily, every other day, 2-4 times a week, 3-5 times a week, weekly, biweekly, monthly, or bimonthly for about 90 days, for about 6 months, for about 1 year or continuously. In an embodiment, the dosage schedule is once a day for about 14 days. In an embodiment, the dosage schedule is once a day for about 84 days.

In embodiments, the pharmaceutical composition comprises: (i) about 8% ivermectin; (ii) about 27.6% mono- and di-C₈ to C₁₀ fatty acid esters of glycerol; (iii) about 32.2% polysorbate 80; and (iv) about 32.2% vitamin E TPGS.

In an embodiment, the pharmaceutical composition comprises: (i) about 8% ivermectin; (ii) about 27.6% Masester E8120; (iii) about 32.2% polysorbate 80; and (iv) about 32.2% vitamin E TPGS.

In an embodiment, the pharmaceutical composition comprises: (i) about 8% ivermectin; (ii) about 27.6% mono- and di-C₈ to C₁₀ fatty acid esters of glycerol; (iii) about 18.4% polysorbate 80; and (iv) about 46% vitamin E TPGS.

In an embodiment, the pharmaceutical composition comprises: (i) about 8% ivermectin; (ii) about 27.6% Masester E8120; (iii) about 18.4% polysorbate 80; and (iv) about 46% vitamin E TPGS.

In an embodiment, the pharmaceutical composition comprises: (i) about 8% ivermectin; (ii) about 27.6% mono- and di-C₈ to C₁₀ fatty acid esters of glycerol; and (iii) about 64.4% polysorbate 80.

In an embodiment, the pharmaceutical composition comprises: (i) about 8% ivermectin; (ii) about 27.6% Masester E8120; and (iii) about 64.4% polysorbate 80.

In embodiments, the ivermectin comprises a compound of Formula VI (22,23-Dihydroavermectin B1a) and a compound of Formula VII 22,23-Dihydroavermectin B1b). In some embodiments, ivermectin comprises at least about 70% of 22,23-dihydroavermectin B1a and less than about 30% of 22,23-dihydroavermectin B1b. In some embodiments, ivermectin comprises at least about 90% of 22,23-dihydroavermectin B1a and less than about 10% of 22,23-dihydroavermectin B1b.

In an embodiment, a pharmaceutical composition comprising (i) about 8% ivermectin; (ii) about 27.6% Masester E8120; (iii) about 32.2% polysorbate 80; and (iv) about 32.2% vitamin E TPGS is administered daily and continuously at a dose of about 10 mg to 120 mg. In an embodiment, a pharmaceutical composition comprising (i) about 8% ivermectin; (ii) about 27.6% Masester E8120; (iii) about 32.2% polysorbate 80; and (iv) about 32.2% vitamin E TPGS is administered daily and continuously at a dose of about 10 mg to 80 mg. In an embodiment, a pharmaceutical composition comprising (i) about 8% ivermectin; (ii) about 27.6% Masester E8120; (iii) about 32.2% polysorbate 80; and (iv) about 32.2% vitamin E TPGS is administered daily and continuously at a dose of about 10 mg to 60 mg. In an embodiment, a pharmaceutical composition comprising (i) about 8% ivermectin; (ii) about 27.6% Masester E8120; (iii) about 32.2% polysorbate 80; and (iv) about 32.2% vitamin E TPGS is administered daily and continuously at a dose of about 20 mg to 40 mg.

In an embodiment, a pharmaceutical composition comprising (i) about 8% ivermectin; (ii) about 27.6% Masester E8120; (iii) about 32.2% polysorbate 80; and (iv) about 32.2% vitamin E TPGS is administered daily. In an embodiment, a pharmaceutical composition comprising (i) about 8% ivermectin; (ii) about 27.6% Masester E8120; (iii) about 32.2% polysorbate 80; and (iv) about 32.2% vitamin E TPGS is administered daily and continuously. In an embodiment, a pharmaceutical composition comprising (i) about 8% ivermectin; (ii) about 27.6% Masester E8120; (iii) about 32.2% polysorbate 80; and (iv) about 32.2% vitamin E TPGS is administered at a dose of about 10 mg daily and continuously. In an embodiment, a pharmaceutical composition comprising (i) about 8% ivermectin; (ii) about 27.6% Masester E8120; (iii) about 32.2% polysorbate 80; and (iv) about 32.2% vitamin E TPGS is administered at a dose of about 20 mg daily and continuously. In an embodiment, a pharmaceutical composition comprising (i) about 8% ivermectin; (ii) about 27.6% Masester E8120; (iii) about 32.2% polysorbate 80; and (iv) about 32.2% vitamin E TPGS is administered at a dose of about 40 mg daily and continuously. In an embodiment, a pharmaceutical composition comprising (i) about 8% ivermectin; (ii) about 27.6% Masester E8120; (iii) about 32.2% polysorbate 80; and (iv) about 32.2% vitamin E TPGS is administered at a dose of about 60 mg daily and continuously. In an embodiment, a pharmaceutical composition comprising (i) about 8% ivermectin; (ii) about 27.6% Masester E8120; (iii) about 32.2% polysorbate 80; and (iv) about 32.2% vitamin E TPGS is administered at a dose of about 80 mg daily and continuously. In an embodiment, a pharmaceutical composition comprising (i) about 8% ivermectin; (ii) about 27.6% Masester E8120; (iii) about 32.2% polysorbate 80; and (iv) about 32.2% vitamin E TPGS is administered at a dose of about 120 mg daily and continuously.

In an embodiment, the neurological disorder is epilepsy. In some embodiments, the epilepsy refractory or treatment-resistant epilepsy.

In embodiments, the method comprises administering to the subject a pharmaceutical composition disclosed herein, with one or more adjunct therapies, including up to four additional anti-epileptic drugs (AEDs), administered simultaneously, sequentially, and in the same or different dosage form as the composition comprising (i) a compound of any one of Formulas I-VII, (ii) one or more of a compound of any one of Formulas I-VII, or (iii) ivermectin comprising a compound of Formula VI and a compound of Formula VII. The AEDs can include drugs such as lorazepam, cenobamate, brivaracetam, striripentol, acetazolamide, phenytoin, sodium valproate, buccal midazolam, felbarnate, clobazam, permpanel, tiagabine, rufinamide, levetiracetam, larnotrigine, pregabalin, primidone, gabapentin, nitrazepam, phenobarbital, clonazepam, vigabatrin, carbamazepine, topiramate, oxcarbazepine, rectal diazepam, lacosamide, ethosuximide, eslicarbazepine acetate, and/or zonisamide. Non-pharmaceutical anti-epileptic therapies may also be administered concomitantly, including implantable devices such as vagal nerve stimulators, responsive neurostimulators, and deep brain stimulators.

### EXAMPLES

### Example 1—Solubility Determination

The active pharmaceutical ingredient (API) used in the following examples, is ivermectin comprising greater than or equal to 90% of 22,23-dihydroavermectin B1a and less than 10% of 22,23-dihydroavermectin B1b. The solubility assessments were performed by continually adding a known amount of the API into each individual excipient and letting the excipient reach solubility equilibrium via API saturation (visible solid observed). The experiments were conducted at ambient conditions (25° C.) and were left to mix on a stir plate at 500 RPM for at least 72 hours. Subsequently, samples were centrifuged and filtered via a 0.45 μm PVDF membrane filter (Millipore Durapore) and filtrate was assayed per United States Pharmacopeia (USP) ivermectin monograph. Quantitative composition of placebo vehicle and the corresponding ivermectin initial solubility results are reported in Table 1. Due to the melting point of Vitamin E TPGS (VE TPGS) at 38° C., the solubility study in VE TPGS was conducted at 40° C. on a hot plate following same procedure as above.

TABLE 1
Ivermectin Solubility Determination at 25° C.

| Excipient | Solubility (mg/g) | Rank |
|---|---|---|
| Maisine CC | 60.17 | 10 |
| Capryol 90 | 150.92 | 2 |
| LauroglycolTM FCC | 71.96 | 9 |
| PEG 400 | 112.92 | 4 |
| Masester E8120 | 164.06 | 1 |
| Labrafil M 2125 CS | 35.32 | 14 |
| Tween 80 | 88.26 | 7 |
| Labrasol ALF | 126.36 | 3 |
| Kolliphor EL | 95.85 | 6 |
| Vitamin E TPGS | 82.98 | 8 |
| Soybean oil | 6.72 | 18 |
| Olive oil | 4.71 | 19 |
| Fractionated Coconut oil | 16.55 | 16 |
| Triacetin | 58.75 | 11 |
| Oleic acid | 48.58 | 13 |
| Medium Chain Triglyceride | 16.49 | 17 |
| Span 20 | 97.66 | 5 |
| Span 80 | 54.04 | 12 |
| Glycerin | <0.1 | 20 |
| Propylene glycol | 25.61 | 15 |

### Example 2—Excipient Compatibility Study

An excipient compatibility study was initiated with selected excipients ranging from oils to surfactants based on equilibrium solubility results (Table 2). The study was designed to provide an ivermectin solution at approximately 5% in selected excipients as in Table 2 with the exception of excipients that the equilibrium solubility are below 5%, namely fractionated coconut oil and oleic acid at approximately 1%. After solutions were prepared using a hand-held homogenizer (Model IKAT10 Ultra-Turrax), each excipient sample was aliquoted in amber vials, approximately 10% water was added in each excipient aliquot, and bench scale gel stripe OET-004037 was also made and added in a separate set of aliquot samples at approximately 1:1 w/w.

All samples were stored at 40° C./75% relative humidity (RH) condition (Model: Lunaire environmental chamber) and tested by developmental reversed phase HPLC method for Assay/Related Substances at T=0, 2 weeks, and 1 month. Results are summarized in Table 3 and Table 4.

Oily vehicles including Maisine CC, fractionated coconut oil, and oleic acid had good chemical compatibility with the API, among which Maisine CC has the highest equilibrium solubility, therefore Maisine CC was chosen to be studied further as an oily vehicle. Tween 80, Labrasol ALF and Vitamin E TPGS categorized as surfactant were compared and Vitamin E TPGS showed the best chemical compatibility with the API with minimum increase in impurities over time, followed by Tween 80 having less stability over time. Both Vitamin E TPGS and Tween 80 were selected as emulsifiers to be used in formulation development. Due to the high solubility in Masester E8120, Masester E8120 was selected for further evaluation for formulation development as solubilizer and emulsifer. PEG 400, as a co-solvent was not considered for further formulation development, due to its poor chemical compatibility.

TABLE 2
Excipients list for Compatibility Study and Actual API w/w %

| | |
|---|---|
| | % w/w |
| Excipient Name | (Corrected for API purity) |

Oily Vehicle

| | |
|---|---|
| Maisine CC | 4.54% |
| Fractionated coconut oil | 0.91% |
| Oleic acid | 0.95% |

Surfactant

| | |
|---|---|
| Tween 80 | 4.62% |
| Labrasol ALF | 4.70% |
| Vitamin E TPGS | 4.61% |

Mixed Glycerides

| | |
|---|---|
| Masester E8120 | 4.63% |

Co-solvent

| | |
|---|---|
| PEG 400 | 4.61% |

TABLE 3
Summary of Excipient Compatibility Study (Assay)

| | Chromatographic purity by HPLC (% area) | | |
|---|---|---|---|
| Excipients | T = 0 | T = 2 weeks | T = 1 months |

Sample: Control

| | | | |
|---|---|---|---|
| Maisine CC | 97.13 | 96.00 | 96.82 |
| Tween 80 | 97.53 | 91.87 | 88.45 |
| Labrasol ALF | 97.74 | 89.16 | 81.35 |
| Vitamin E TPGS | 97.74 | 97.43 | 97.37 |
| Fractionated coconut oil | 95.61 | 97.94 | N/A |
| Oleic acid | 99.60 | 83.55 | N/A |
| Masester E8120 | 97.58 | 96.81 | 97.06 |
| PEG 400 | 97.58 | 89.36 | N/A |

Sample: 10% water

| | | | |
|---|---|---|---|
| Maisine CC | N/A | 95.90 | 96.83 |
| Tween 80 | N/A | 93.02 | 91.18 |
| Labrasol ALF | N/A | 93.91 | 89.91 |
| Vitamin E TPGS | N/A | 96.99 | 97.46 |
| Fractionated coconut oil | N/A | 97.83 | N/A |
| Oleic acid | N/A | 86.02 | N/A |
| Masester E8120 | N/A | 95.11 | 91.68 |
| PEG 400 | N/A | 94.35 | N/A |

Sample: Gel Stripe (1:1 w/w)

| | | | |
|---|---|---|---|
| Maisine CC | N/A | 96.15 | 96.94 |
| Tween 80 | N/A | 93.97 | 93.51 |
| Labrasol ALF | N/A | 97.11 | 96.49 |
| Vitamin E TPGS | N/A | 97.50 | 97.74 |
| Fractionated coconut oil | N/A | 94.42 | N/A |
| Oleic acid | N/A | 83.73 | N/A |
| Masester E8120 | N/A | 97.35 | 97.31 |
| PEG 400 | N/A | 96.92 | N/A |

TABLE 4
Results Summary for Compatibility Study of Excipient
Samples (Total impurities). % adjusted area =
Total Impurities % excluding API impurities %

| | Total Impurities % (% adjusted area) | | |
|---|---|---|---|
| Excipients | T = 0 | T = 2 weeks | T = 1 months |

Sample: Control

| | | | |
|---|---|---|---|
| Maisine CC | 0.58 | 0.35 | 0.71 |
| Tween 80 | 0.16 | 4.86 | 10.55 |
| Labrasol ALF | <LOQ | 8.67 | 20.49 |
| Vitamin E TPGS | <LOQ | <LOQ | 0.11 |
| Fractionated coconut oil | <LOQ | <LOQ | N/A |
| Oleic acid | <LOQ | 17.27 | N/A |
| Masester E8120 | 0.17 | 0.58 | 0.45 |
| PEG 400 | 0.10 | 8.54 | N/A |

Sample: 10% water

| | | | |
|---|---|---|---|
| Maisine CC | N/A | 0.45 | 0.69 |
| Tween 80 | N/A | 3.53 | 6.94 |
| Labrasol ALF | N/A | 3.46 | 8.76 |
| Vitamin E TPGS | N/A | 0.42 | <LOQ |
| Fractionated coconut oil | N/A | <LOQ | N/A |
| Oleic acid | N/A | 9.89 | N/A |
| Masester E8120 | N/A | 2.27 | 6.60 |
| PEG 400 | N/A | 3.15 | N/A |

Sample: Gel Stripe (1:1 w/w)

| | | | |
|---|---|---|---|
| Maisine CC | N/A | 0.33 | 0.58 |
| Tween 80 | N/A | 2.43 | 4.22 |
| Labrasol ALF | N/A | 0.30 | 1.06 |
| Vitamin E TPGS | N/A | <LOQ | <LOQ |
| Fractionated coconut oil | N/A | 3.28 | N/A |
| Oleic acid | N/A | 11.82 | N/A |
| Masester E8120 | N/A | 0.11 | 0.18 |
| PEG 400 | N/A | 0.50 | N/A |

### Example 3—Fiber Optic Dispersion Study

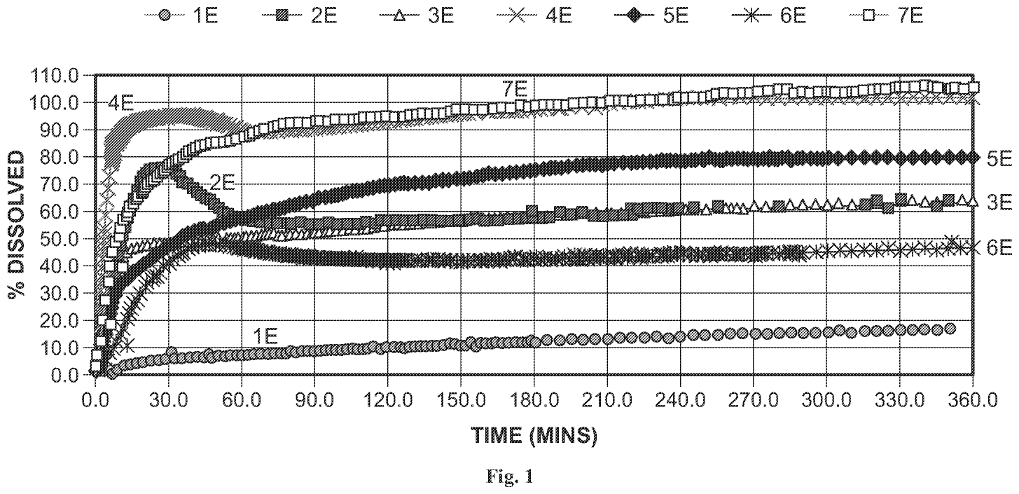

Fiberoptic Dispersion in fasted state simulated intestinal fluid (FaSSIF) and FaSSIF-V2 were performed using a Distec dissolution system 2500 and PIONfiber optic dissolution system to collect real-time dissolution data and data processing. With a target of 20 mg/capsule and based on the compatibility studies, a range of formulations (Table 5) were designed for dispersibility evaluations. FaSSIF medium was chosen to mimic intestinal environment after ivermectin is ingested orally. Dispersion analysis was conducted firstly in 500 mL Fasted State Simulated Intestinal Fluid (pH˜6.5) at 50 rpm paddle speed (USP <711> Apparatus II) for screening various excipient combinations and compositions for approximately 6 hours. Results were plotted as a graph shown in FIG. 1.

FaSSIF-V2 was used later to confirm dispersibility of formulations 7E and 9E as they have shown higher dispersibility than other formulation evaluated from the first round of the dispersion study.

Kinetic solubility in FaSSIF and FaSSIF-V2 for both 7E and 9E reached 800% release at T=30 minutes suggesting an immediate release profile. No drug precipitation was observed indicating good dispersibility of both formulations and ability to maintain solubilization of API with low risk of API precipitation once ingested into GI tract. Data processing using Aupro.

TABLE 5
Quantitative Compositions of Formulations in Dispersion Study

| Formulation # | Components | % w/w |
|---|---|---|
| 1E | Ivermectin | 10.0% |
| | Masester E8120 | 90.0% |
| | Total | 100% |
| 2E | Ivermectin | 5.0% |
| | Maisine CC | 47.5% |
| | Tween 80 | 47.5% |
| | Total | 100% |
| 3E | Ivermectin | 10.0% |
| | Masester E8120 | 63.0% |
| | VE TPGS | 27.0% |
| | Total | 100% |
| 4E | Ivermectin | 8.0% |
| | Masester E8120 | 27.6% |
| | Tween 80 | 64.4% |
| | Total | 100% |
| 5E | Ivermectin | 10.0% |
| | Masester E8120 | 45.0% |
| | Tween 80 | 18.0% |
| | VE TPGS | 27.0% |
| | Total | 100% |
| 6E | Ivermectin | 5.0% |
| | Maisine CC | 47.5% |
| | Tween 80 | 19.0% |
| | VE TPGS | 28.5% |
| | Total | 100% |
| 7E | Ivermectin | 8.0% |
| | Masester E8120 | 27.6% |
| | Tween 80 | 32.2% |
| | VE TPGS | 32.2% |
| | Total | 100% |
| 9E | Ivermectin | 8.0% |
| | Masester E8120 | 27.6% |
| | Tween 80 | 18.4% |
| | VE TPGS | 46.0% |
| | Total | 100% |

### Example 4—Analysis of Prototype Formulations

7E and 9E were selected as prototype formulations following the kinetic solubility study where both formulations showed good dispersibility in simulated intestinal fluids. A 12-month informal stability study was conducted to challenge these two formulations at 40° C./75% RH in a Lunaire environmental chamber and with the addition of 5% water and gel stripes (OET-004037) in separate vials to mimic softgel water migration and the impact of gel on formulation in softgel dosage form, respectively. The formulation control samples (without water and gel stripes) were stored in 8 oz amber Boston round bottles to mimic preliminary intended packaging for first in human clinical study (SAD/MAD study). All other samples were stored in amber glass vials. The Assay/RS results are summarized in Table 6 and Table 7, analyzed by a developmental HPLC method.

There was no precipitation nor phase separation observed at T=0 to 1 month at 40° C./75% RH in all sample vials. There was light slurry observed in the T=3-month water added sample in 7E which indicates minor API precipitation with 5% water addition possibly due to polymorphism change of API. However, water added samples is a simulation and not a true representation of water migration in softgels. The minor precipitation in this sample is inconclusive and deemed to be minor in terms of formulation stability.

TABLE 6
Results Summary for Informal Stability
Study of Prototype Formulations (Assay)

| Prototype | Chromatographic purity by HPLC (% LC) | | | |
|---|---|---|---|---|
| Formulation | T = 0 | T = 2 weeks | T = 1 months | T = 3 months |

Sample: Control

| | | | | |
|---|---|---|---|---|
| 7E | 99.22 | 100.52 | 101.10 | 94.02 |
| 9E | 99.59 | 98.32 | 98.34 | 98.68 |

Sample: 10% water

| | | | | |
|---|---|---|---|---|
| 7E | N/A | 97.00 | 96.29 | 98.07 |
| 9E | N/A | 94.24 | 92.82 | 101.45 |

Sample: Gel Stripe (1:1 w/w)

| | | | | |
|---|---|---|---|---|
| 7E | N/A | 97.51 | 97.55 | 94.20 |
| 9E | N/A | 96.62 | 96.23 | 104.00 |

TABLE 7
Results Summary for Informal Stability Study of
Prototype Formulations (Related Substances)

| Prototype | Chromatographic purity by HPLC (% area) | | | |
|---|---|---|---|---|
| Formulation | T = 0 | T = 2 weeks | T = 1 months | T = 3 months |

Sample: Control

| | |
|---|---|
| 7E | 6.56 |
| 9E | 6.05 |

Sample: 10% water

| | |
|---|---|
| 7E | 5.67 |
| 9E | 5.33 |

Sample: Gel Stripe (1:1 w/w)

| | |
|---|---|
| 7E | 3.25 |
| 9E | 3.55 |

### Example 5—Freeze-Thaw Study

Prototype formulations 7E and 9E were challenged in a freeze-thaw study for physical and chemical stability from −15° C. to 40° C. in a ChinaCell environmental test chamber for a 7-day period of time, 24-hour cycle. There was no precipitation nor phase separation observed from two samples vials at day 7. Chemical stability (Assay) is summarized in Table 8. It can be concluded that both formulations are stable under extreme temperature fluctuation stress.

TABLE 8
Results Summary for Freeze-thaw Study
of Prototype Formulations (Assay)

Chromatographic purity by HPLC (% area)

| Prototypes | T = 0 | T = day 7 |
|---|---|---|
| 7E | 99.22 | 101.3 |
| 9E | 99.59 | 98.80 |

### Example 6—Pharmacokinetic Profiles of Ivermectin B1a in Sprague Dawley Rats Following IV and Oral Administration

Thirty-six male Sprague Dawley rats were randomly assigned into four groups, with nine male rats in each group. All animals in all groups are fasted overnight prior to the experimental date.

Rats in Group 1 were administered ivermectin API solubilized in DMSO, solutol, and saline (n=9) administered intravenously (IV). Rats in Group 2 were administered 2 mg/kg Stromectol crushed and solubilized in water (n=9) via oral gavage. Stromectol is formulated by the manufacturer as a tablet comprising 3 mg ivermectin comprising at least 90% 22,23-dihydroavermectin B1a and less than 10% 22,23-dihydroavermectin B1b. Rats in Group 3 were administered 2 mg/kg Ivomec in water (n=9) via oral gavage. Ivomec is formulated by the manufacturer as a 1% ivermectin solution. Rats in Group 4 were administered 2 mg/kg prototype formulations 7E (“7E”) in water (n=9) via oral gavage.

Blood samples were collected from 6 of 9 animals per group at 0.5, 1, 2, 3, 4, 6, 8, 24, 72, 120, 168 hours post-dose. The collected plasma samples were analyzed for the concentration of Ivermectin B1a by a validated LC-MS/MS method. Pharmacokinetic parameters were calculated by the concentration data of Ivermectin B1a in plasma samples using non-compartment model of Phoenix WinNonlin 7.0 software.

The remaining 3 of 9 animals per group were sacrificed 4 hours after study product administration. Blood samples were collected via jugular vein or other suitable vein. Brains were removed, rinsed with saline, dried with filter paper and weighed immediately. The brain samples were homogenized and the samples were analyzed for the concentration of Ivermectin B1a by a LC-MS/MS method per FDA's guidelines. The brain samples and plasma sample ratios were calculated for the BBB penetration percentages.

### Results

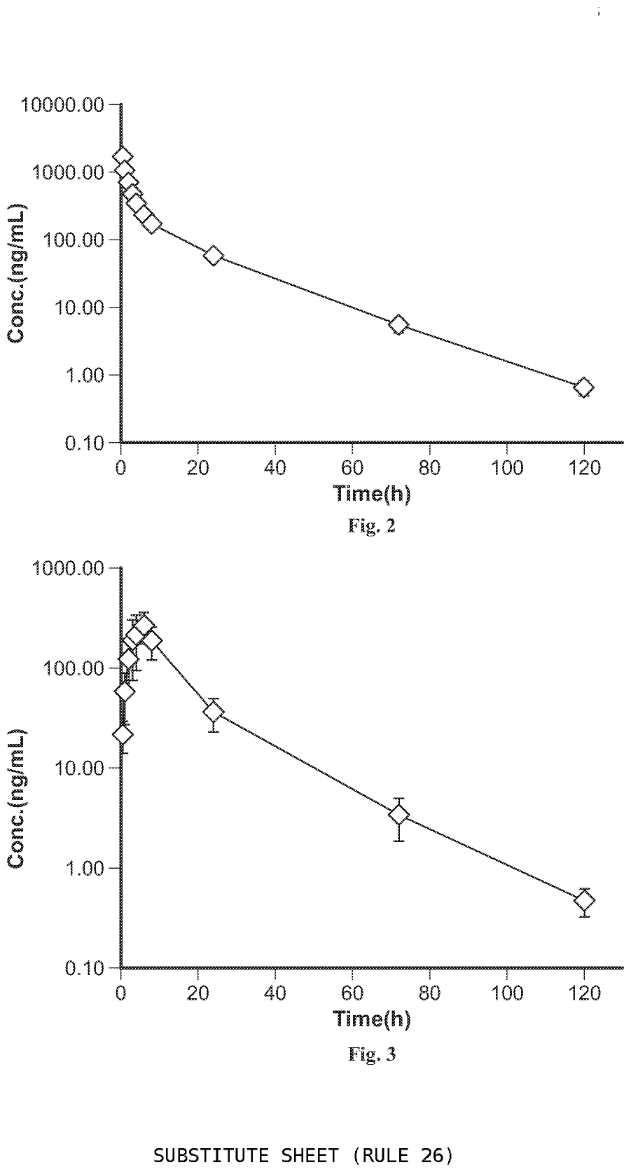

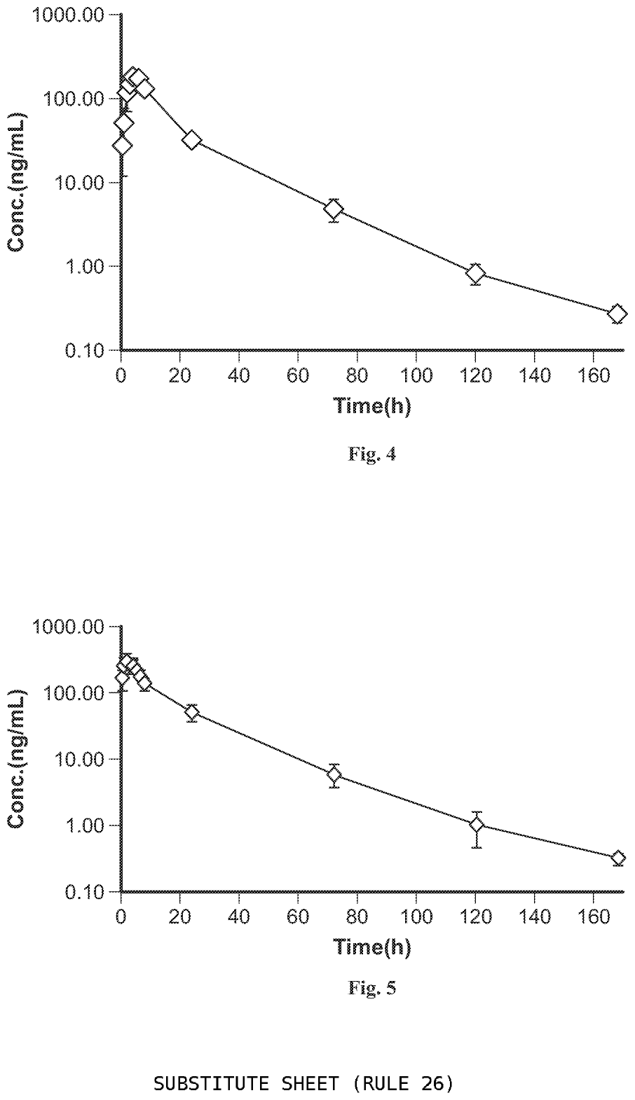

The main pharmacokinetic parameters of Ivermectin B1a in SD rat plasma after single administration of Ivermectin API, Stromectol, Ivomec, and 7E are summarized in Table 9 and FIGS. 2-5.

TABLE 9
Main Pharmacokinetic Parameters of Ivermectin B1a in SD Rat Plasma After Single Intravenous
Administration of Ivermectin (API) or Oral Administration of Stromectol, Ivomec, and
7E (Mean ± SD). Dose level: 2 mg/kg. Fating status: Fasting. Sex: Male. n = 6.

| Group | Route of | T_{1/2} | Tₘₐₓ | Cₘₐₓ | AUC₍₀₋ₜ₎ | AUC_{(0-∞)} | MRT₍₀₋ₜ₎ | MRT_{(0-∞)} |
|---|---|---|---|---|---|---|---|---|
| No. | Dosin | h | h | ng/ml | h*ng/ml | h*ng/ml | h | h |
| 1 | IV | 14.82 | 0.50 ± | 1722.67 | 8259.31 ± | 8273.53 ± | 10.58 ± | 10.81 ± |
| API | | | 0.0 | | 1032.9 | 1034.0 | 0.83 | 0.85 |
| 2 | PO | 12.85 | 5.67 ± | 278.4 ± | 4220.94 ± | 4244.6 ± | 13..52 ± | 14.11 ± |
| Stromecto | | | 0.8 | 109.3 | 1594.2 | 1589.3 | 1.49 | 1.35 |
| 3 | PO | 18.49 | 4.83 ± | 190.42 ± | 3396.36 ± | 3410.59 ± | 17.26 ± | 17.85 ± |
| Ivomec | | | 1.3 | 25.36 | 353.94 | 354.24 | 2.75 | 2.63 |
| 4 | PO | 17.15 | 2.17 ± | 306.28 ± | 4677.49 ± | 4691.6 ± | 16.71 ± | 17.15 ± |
| 7E | | | 0.7 | 61.41 | 920.69 | 916.77 | 2.27 | 2.37 |

Group 1: After a single IV injection of 2 mg/kg Ivermectin API in male SD rats with fasting, the AUC₍₀₋ₜ₎ of Ivermecin B1a in plasma was 8259.31±1032.95 hr*ng/ml, the T_{1/2} were 14.82±0.72 hr.

Group 2: After a single PO administration of 2 mg/kg Stromectol in male SD rats with fasting, the AUC₍₀₋ₜ₎ of Ivermecin B1a in plasma was 4220.94±1594.22 hr*ng/ml, the T_{1/2} were 12.85±2.16 hr.

Group 3: After a single PO administration of 2 mg/kg Ivomec in male SD rats with fasting, the AUC₍₀₋ₜ₎ of Ivermecin B1a in plasma was 3396.36±353.94 hr*ng/ml, the T_{1/2} were 18.49±1.89 hr.

Group 4: After a single PO administration of 2 mg/kg 7E in male SD rats with fasting, the AUC₍₀₋ₜ₎ of Ivermecin B1a in plasma was 4677.49±920.69 hr*ng/ml, the T_{1/2} were 17.15±13.14 hr.

The ratio between the concentration of Ivermectin B1a in brain and the concentration of Ivermectin B1a in the plasma were calculated and summarized in Table 10.

TABLE 10
Ratio of Ivermectin B1a in SD Rat Plasma and Brain After Single
Intravenous Administration of Ivermectin (API) or Oral Administration
of Stromectol, Ivomec, and 7E (Mean ± SD). Dose level:
2 mg/kg. Fating status: Fasting. Sex: Male. n = 3.

| | | Conc. of | Conc. of | |
|---|---|---|---|---|
| | Route | ivermectin | ivermectin | Brain:Plasma |
| Group | of | B1a in brain | B1a in plasma | Ratio |
| No. | Dosing | ng/ml | ng/ml | — |
| 1 | IV | 79.26 ± | 462.58 ± | 0.171 ± |
| API | | 11.97 | 43.49 | 0.010 |
| 2 | PO | 13.97 ± | 299.25 ± | 0.046 ± |
| Stromectol | | 4.97 | 71.61 | 0.006 |
| 3 | PO | 8.08 ± | 235.98 ± | 0.033 ± |
| Ivomec | | 3.81 | 63.50 | 0.008 |
| 4 | PO | 12.84 ± | 301.27 ± | 0.043 ± |
| 7E | | 0.36 | 46.06 | 0.005 |

Group 1: After a single IV injection of 2 mg/kg Ivermectin API in male SD rats with fasting, the ratio between the concentration of Ivermectin B1a in the brain and the concentration of Ivermectin B1a in the plasma was 0.171±0.010.

Group 2: After a single PO administration of 2 mg/kg Stromectol in male SD rat with fasting, the ratio between the concentration of Ivermectin B1a in the brain and the concentration of Ivermectin B1a in the plasma was 0.046±0.006.

Group 3: After a single PO administration of 2 mg/kg Ivomec in male SD rats with fasting, the ratio between the concentration of Ivermectin B1a in the brain and the concentration of Ivermectin B1a in the plasma was 0.033±0.008.

Group 4: After a single PO administration of 2 mg/kg 7E in male SD rats with fasting, the ratio between the concentration of Ivermectin B1a in the brain and the concentration of Ivermectin B1a in the plasma was 0.043±0.005.

### Conclusion

After a single fasting oral administration of 2 mg/kg Stromectol and 7E there was no significant difference in Cₘₐₓ (Stromectol vs 7E 1:1.10) or AUC₀₋ₜ(Stromectol vs 7E 1:1.11).

### Example 7—Modulation of Pentylenetetrazol (PTZ)-Induced Seizures by Liquid Solution of Formulation 7E in Rats

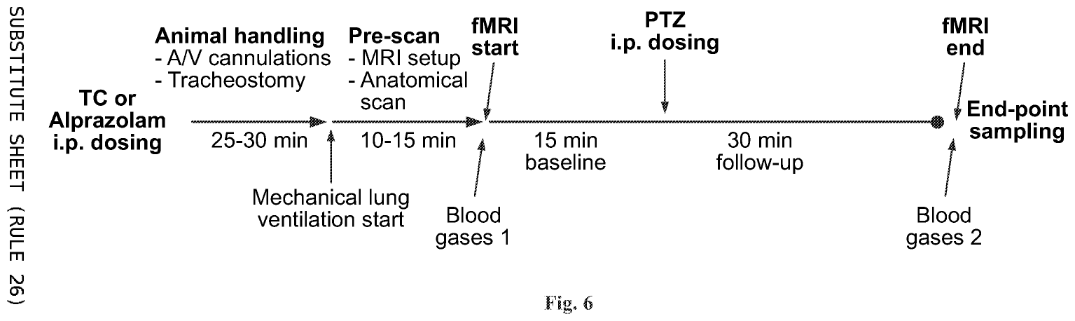

Functional MRI (fMRI) was performed using a high-field preclinical MRI system in anesthetized male Wistar rats in order to determine the modulation of pentylenetetrazol (PTZ)-induced ictal brain activity by chronic treatment with test compound (TC). Animals treated with vehicle and 3 mg/kg Alprazolam served as controls. A schematic of the study design can be seen in FIG. 6. The TC was a liquid solution of formulation 7E at either 2 mg/kg or 4 mg/kg. Various physiological parameters were also measured throughout the experiment including body weight, CO₂ levels, blood pH, and heart rate.

### Results

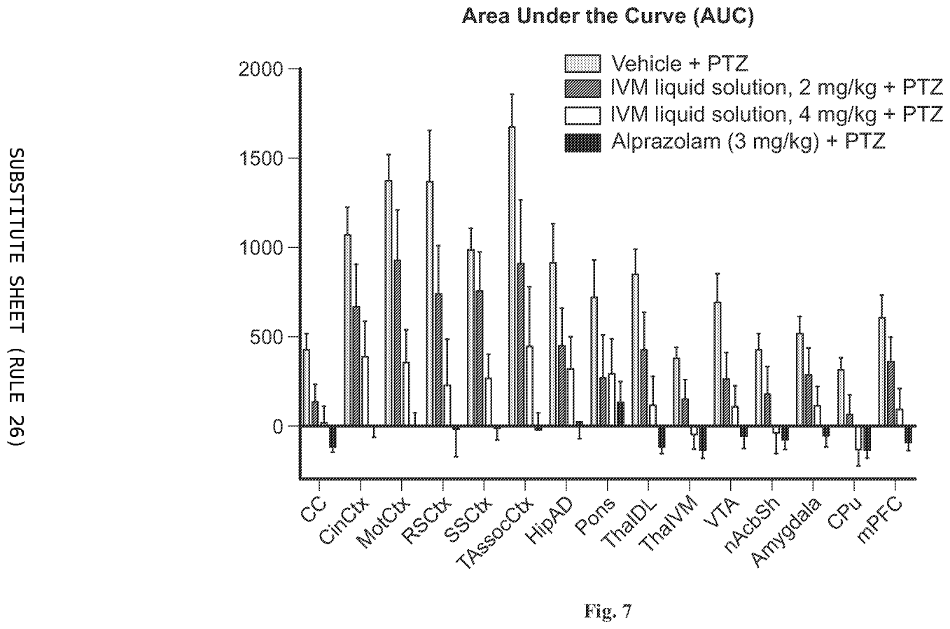

Area under the curve was calculated for each brain region and treatment as shown in FIG. 7. In each brain area, a significant decrease in (PTZ)-induced ictal brain activity was seen with both 2 mg/kg and 4 mg/kg dosages of the ivermectin liquid solution having formulation 7E, as compared to the control (vehicle).

### Conclusion

Physiological parameters measured with arterial blood sampling and pulse oximetry indicated good cardiovascular and respiratory stability of the subjects throughout the functional imaging experiments. Positive control compound (Alprazolam, 3 mg/kg) effectively cancelled PTZ-induced brain activity with high statistical significance in all tested brain regions.

Both doses of the liquid solution of formulation 7E demonstrated significant modulation of the PTZ-induced BOLD signal change in several brain areas (Striatum, Hippocampus, VTA, cortical areas) with dose-dependent manner, shortening or attenuating the activity duration. The higher IVM dose 4 mg/kg induced significant modulation in all studied brain regions attenuating the PTZ-response.

### Example 8—Patient Dosed Phase 1 Study

To evaluate safety, tolerability, PK, and high-fat food effects on formulation 7E, a Phase 1 double-blind, placebo-controlled, randomized (3:1), dose-finding study was performed in healthy subjects and its effects on quality of life was assessed. A single ascending dose (SAD) portion of the study was followed by a multiple ascending dose (MAD) cohorts at 10, 20, 40, 80 and 120 mg of formulation 7E once a day for 14 days. The study population were healthy individuals over 18-65 years of age.

### SAD Portion of the Study

Participants were admitted to the clinical research center the day before dosing for a baseline assessment, including an eligibility review, physical exam, neurological exam, laboratory evaluations, an EKG, and pupillometry. Subjects followed a fasting dose protocol and were given a single dose administration of the study product followed by serial PK, vital signs, and safety assessments including pupillometry and neurological exams over 120 hours. Additionally, PK sample collection occurred at the following time points: pre-dose (within 2 hours prior to dosing) and at 0.5, 1, 2, 3, 4, 5, 6, 8, 10, 12, 16, 24, 36, 48, 72, and 120 hours post-dose). Each subsequent SAD cohort (see, Table 11) begun 72 hours after the last subject in the previous cohort was dosed, as long as fewer than 2 of the participants in that cohort experienced a dose limiting toxicity (DLT), as ivermectin toxicity manifests within 24 hours of dosing. Participants in the 40 mg cohort of the SAD portion of the study were not discharged on day 6 and instead continued onto a fed sub-study, with the fed dose administered 10 days after the fasted dose in conjunction with a high-fat meal. Evaluations following the fed dose were identical to those after the fasted SAD doses and continued through 120 hours.

Two vials of whole blood samples for PD measurements were collected pre-dose and 24 hours after the single dose for exploratory biomarker research.

### MAD Portion of the Study (14-Day Study, One Dose Per Day)

Each MAD cohort 10 mg-80 mg (see, Table 11) begun dosing the day after the SAD cohort at the dose above had completed dosing and had been monitored for 72 hours, provided stopping criteria were not met in the SAD study or in a lower dose MAD cohort. If stopping criteria were not yet met, the 120 mg MAD cohort begun after the last subject in the 80 mg cohort had been dosed through day 5, as steady state was estimated to occur around 4 days.

Volunteers underwent screening and baseline assessments as described above for the SAD portion of the study. Subjects followed a fasting dose protocol on days 1, 2, and 14, and were given a single daily administration of the study product for 14 days. After dosing on days 1, 7 and 14, vital signs and safety assessments including pupillometry and neurological exams were performed. These and additional safety assessments were also performed on day 19. PK sample collection occurred at the following time points after Day 1 and Day 14 dosing: pre-dose (within 2 hour prior to dosing) and at 0.5, 1, 2, 3, 4, 5, 6, 8, 10, 12, 16, 24, 36, 48, 72, and 120 hours (pre-dose) after the initial day 1 dose. Blood for PK was also drawn pre-dose on days 5, 7, 8, 9, 10, 12, and 14. If two participants in a cohort experienced a DLT, that cohort was stopped as were any overlapping cohorts at a higher dose.

Two vials of whole blood samples for PD measurements were collected pre-dose and on Day 15 (24 hours after a subject's last dose of study drug in the MAD study) for exploratory biomarker research.

TABLE 11
Phase I Dose Cohorts

| Study | Dose | No. of | Proposed Doses (May Differ |
|---|---|---|---|
| Portion | Cohort | Subjects | Based on CRM Output) |
| SAD | 1 | 8 | Formulation 7E gelcap 10 mg (n = 6) |
| | | | vs placebo (n = 2) |
| SAD | 2 | 8 | Formulation 7E gelcap 20 mg (n = 6) |
| | | | vs placebo (n = 2) |
| SAD | 3 | 8 | Formulation 7E gelcap 40 mg (n = 6) |
| | | | vs placebo (n = 2) |
| SAD | 4 | 8 | Formulation 7E gelcap 80 mg (n = 6) |
| | | | vs placebo (n = 2) |
| SAD | 5 | 8 | Formulation 7E gelcap 120 mg (n = 6) |
| | | | vs placebo (n = 2) |
| MAD | 1 | 8 | Formulation 7E gelcap 10 mg (n = 6) |
| | | | vs placebo (n = 2) |
| MAD | 2 | 8 | Formulation 7E gelcap 20 mg (n = 6) |
| | | | vs placebo (n = 2) |
| MAD | 3 | 8 | Formulation 7E gelcap 40 mg (n = 6) |
| | | | vs placebo (n = 2) |
| MAD | 4 | 8 | Formulation 7E gelcap 80 mg (n = 6) |
| | | | vs placebo (n = 2) |
| MAD | 5 | 8 | Formulation 7E gelcap 120 mg (n = 6) |
| | | | vs placebo (n = 2) |
| Food | 4* | 8 | Formulation 7E gelcap 40 mg (n = 6) |
| effect | | | vs placebo (n = 2) |

*Formulation 7E gelcap 40 mg (or MTD, whichever is lower) (n = the 8 from the 40 mg or MTD SAD cohort)

### Example 9—Patient Dosed Phase 2 Study (Daily, 12 Weeks)

To evaluate preliminary efficacy in terms of overall seizure reduction, seizure reduction by seizure type (focal, generalized, and unknown), reduction of generalized tonic-clonic seizures and focal to generalized tonic-clonic seizures, seizure freedom over time, tolerability, and impacts suicidality of formulation 7E, a double-blind, placebo-controlled, randomized (4:1), a Phase II safety and dose-finding study of adjunctive composition 7E is performed. The population are subjects ages 18 years to 70 years who have been diagnosed with epilepsy according to International League Against Epilepsy (ILAE) Classification of the Epilepsies 2017 criteria (ILAE 2017) and who are uncontrolled on one to four concomitant antiepileptic drugs (AEDs) at optimal stable dosages for >4 weeks prior to screening and throughout the treatment period.

Cohorts (see, Table 12) are dosed sequentially beginning with Dose Cohort 1. This study initially randomizes a total of 10 subjects into the first dosing cohort (10 mg daily, 4:1 active to placebo) for 12 weeks. Safety data is reviewed after 14 days to determine whether the next cohort can be opened. Dosing continues through a continuous reassessment method (CRM) with oversight of the Safety Review Committee (SRC) after each cohort until 10 subjects have completed 14 days of dosing at 60 mg QD, or until a total of 10 subjects have received the same active dose for 14 days and this dose is identified as the MTD. In each dosing cohort, subjects with no DLTs during the initial 14 days of treatment continue with dosing through 12 weeks, as long as they do not experience a DLT, otherwise meet withdrawal criteria, or decide to withdraw from treatment.

Evaluations of PK parameters together with safety, efficacy, and concomitant anti-epileptic drugs (AED) are made after 2, 4, 8, and 12 weeks of treatment by an unblinded study statistician and provided to a sponsor-designated study physician for review. This review will be performed separately from the review for dose escalation, which is based on DLT development.

TABLE 12
Phase II Dose Cohorts, Estimated

| | No. of | Proposed Doses (May Differ |
|---|---|---|
| Dose Cohort | Subjects | Based on CRM Output) |
| 1 | 10 | Formulation 7E gelcap 10 mg (n = 8) |
| | | or matching placebo (n = 2) |
| 2 | 10 | Formulation 7E gelcap 20 mg (n = 8) |
| | | or matching placebo (n = 2) |
| 3 | 10 | Formulation 7E gelcap 40 mg (n = 8) |
| | | or matching placebo (n = 2) |
| 4 | 10 | Formulation 7E gelcap 60 mg (n = 8) |
| | | or matching placebo (n = 2) |

We claim:

1. A pharmaceutical composition comprising:
(i) about 1% to about 15% ivermectin comprising a compound of Formula VI and a compound of Formula VII: 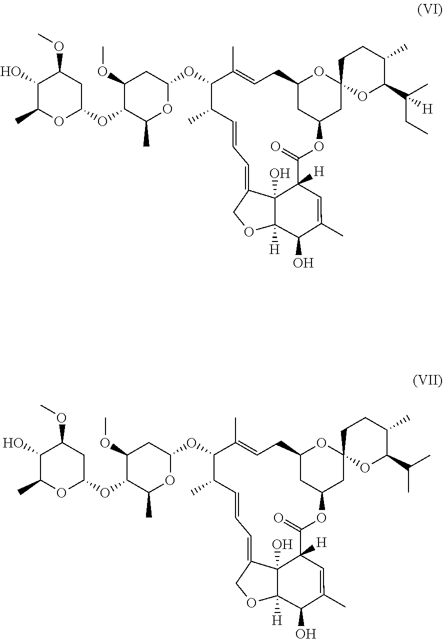 [SMILES: CC[C@H](C)[C@H]1O[C@]2(CC[C@@H]1C)C[C@@H]1C[C@@H](C/C=C(\C)[C@@H](O[C@H]3C[C@H](OC)[C@@H](O[C@H]4C[C@H](OC)[C@@H](O)[C@H](C)O4)[C@H](C)O3)[C@@H](C)/C=C/C=C3\CO[C@@H]4[C@H](O)C(C)=C[C@@H](C(=O)O1)[C@]34O)O2 ; CO[C@H]1C[C@H](O[C@H]2[C@H](C)O[C@@H](O[C@@H]3/C(C)=C/C[C@@H]4C[C@@H](C[C@]5(CC[C@H](C)[C@@H](C(C)C)O5)O4)OC(=O)[C@@H]4C=C(C)[C@@H](O)[C@H]5OC/C(=C\C=C\[C@@H]3C)[C@]54O)C[C@@H]2OC)O[C@@H](C)[C@@H]1O]
(ii) about 20% to about 40% of a first surfactant comprising one or more of:
(a) mono-, di-, and/or tri-fatty acid esters of glycerol;
(b) mono- and/or di-fatty acid esters of 1,2-propylene glycol; and
(c) mono- and/or di-fatty acid esters of polyethylene glycol wherein the fatty acids are selected from C₆ to C₁₀ fatty acids; and
(iii) about 15% to about 70% of a second surfactant selected from one or more of a polysorbate surfactant and/or a fatty acid ester of sorbitan.

2. The pharmaceutical composition of claim 1, wherein the ivermectin comprises at least about 70% of a compound of Formula VI and less than about 30% of a compound of Formula VII.

3. The pharmaceutical composition of claim 2, wherein the ivermectin comprises at least about 90% of a compound of Formula VI and less than about 10% of a compound of Formula VII.

4. The pharmaceutical composition of any one of the preceding claims, wherein the pharmaceutical composition comprises about 3% to about 12% of ivermectin.

5. The pharmaceutical composition of any one of claims 1 to 4, wherein the fatty acids for the first surfactant are selected from C₈ to C₁₀ fatty acids.

6. The pharmaceutical composition of claim 5, wherein the first surfactant comprises mono- and di-fatty acid esters of glycerol.

7. The pharmaceutical composition of any one of claims 1 to 6, wherein the first surfactant is selected from Masester M8120, Capryol 90, Labrasol ALF, and combinations thereof.

8. The pharmaceutical composition of any one of claims 1 to 7, wherein the second surfactant is selected from polysorbate 80 (Tween 80), sorbitan monolaurate (Span 20), and combinations thereof.

9. The pharmaceutical composition of any one of claims 1 to 8 comprising about 25% to about 30% of the first surfactant.

10. The pharmaceutical composition of any one of claims 1 to 9 comprising about 15% to about 20% of the second surfactant.

11. The pharmaceutical composition of any one of claims 1 to 10 comprising about 30% to about 35% of the second surfactant.

12. The pharmaceutical composition of any one of claims 1 to 9 comprising about 60% to about 65% of the second surfactant.

13. The pharmaceutical composition of any one of claims 1 to 11, further comprising about 5% to about 55% vitamin E TPGS; or about 30% to about 50% vitamin E TPGS.

14. The pharmaceutical composition of any one of claims 1 to 4 comprising: (i) about 5% to about 10% ivermectin; (ii) about 25% to about 30% mono- and di-C₈ to C₁₀ fatty acid esters of glycerol; (iii) about 15% to about 30% of a polysorbate surfactant; and (iv) about 30% to about 50% vitamin E TPGS.

15. The pharmaceutical composition of any one of claims 1 to 4 comprising: (i) about 8% ivermectin; (ii) about 27.6% mono- and di-C₈ to C₁₀ fatty acid esters of glycerol; (iii) about 32.2% polysorbate 80; and (iv) about 32.2% vitamin E TPGS.

16. The pharmaceutical composition of any one of claims 1 to 4 comprising: (i) about 8% ivermectin; (ii) about 27.6% Masester E8120; (iii) about 32.2% polysorbate 80; and (iv) about 32.2% vitamin E TPGS.

17. The pharmaceutical composition according to any one of claims 1 to 4 comprising: (i) about 8% ivermectin; (ii) about 27.6% mono- and di-C₈ to C₁₀ fatty acid esters of glycerol; (iii) about 18.4% polysorbate 80; and (iv) about 46% vitamin E TPGS.

18. The pharmaceutical composition of any one of claims 1 to 4 comprising: (i) about 8% ivermectin; (ii) about 27.6% Masester E8120; (iii) about 18.4% polysorbate 80; and (iv) about 46% vitamin E TPGS.

19. A pharmaceutical dosage form comprising a composition of any one of claims 1 to 18 in a gelatin capsule.
